(19) United States
(12) Patent Application Publication
(10) Pub. No.: US 2006/0223993 A1
(43) Pub. Date: Oct. 5, 2006

(54) Colorant compounds, intermediates, and compositions

(76) Inventors: Daniel M. Connor, Inman, SC (US); Larry Trzupek, Greenville, SC (US); Moses Lee, Greenville, SC (US); Daniel Whitehead, Lansing, MI (US)

Correspondence Address: John E. Vick, Jr.;Legal Department, M-495, P.O. Box 1926, Spartaburg, SC, 29304, US

(21) Appl. No.: 11/098,653

(22) Filed: Apr. 1, 2005

(51) Int. Cl. C07D 495/02; C07D 493/02; C07D 471/02; C07D 455/04

(52) U.S. Cl. 540/200; 548/429; 549/45; 549/280; 549/299; 546/80

(57) ABSTRACT

Colorants are disclosed that exhibit high color strength, bright shades, and high thermal stability. Such compounds have found application as colorants for polyethylene terephthalate (“PET”). Potential end uses include disperse dyes, non-warping pigments, decolorizable colorants, and the like. Compounds and methods for synthesis include benzodifuranone related compounds, benzene centered lactones, benzene centered lactams; benzene-centered thiolactones; naphthalene-centered lactones; naphthalene-centered lactams; naphthalene-centered thiolactones; anthraquinone-centered lactones; anthraquinone-centered lactams; anthraquinone-centered thiolactones; anthracene-centered lactones; anthracene-centered lactams; anthracene-centered thiolactones; hetero-aromatic-centered lactones; hetero-aromatic centered lactams and hetero-aromatic centered thiolactone compounds, and the like. Furthermore, resins such as PET or other polymeric resins containing the compounds are disclosed.

### BACKGROUND OF THE INVENTION

Benzodifuranone-based (“BDF” or “BDF's”) colorant compounds are known in the art. U.S. Pat. No. 5,665,150 and J. Soc. Dyers Colour. 110, 1994, p. 178 discloses colorants of the BDF class. A common example of a BDF colorant is shown below. Naphthodifuranone compounds are also known (GB 2,299,811, Dyes and Pigments, 48, 2001, 121-132).

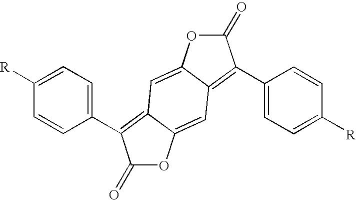

Benzodipyrrole-2-one dyestuffs (U.S. Pat. No. 4,122,087) and benzodithiophene-2-one dyestuffs (JP 09193547) are also known. Dyestuff of the general BDF structure which contain one thiolactone moiety and one lactone moiety and other mixed lactone/thiolactone/lactam moieties are also known (EP 0,033, 583). Two BDFs are known to be commercially available: Sumikaron® Brilliant Red S-BWF by Sumitomo Chemical and Dispersol® Red C-BN by BASF. The main applications for BDF colorants have been as disperse dyes for polyester and other hydrophobic fibers.

U.S. Pat. No. 6,492,533 discloses bismethine benzodifuranone derived colorants. These compounds are useful for applying color to thermoplastics, fibers, and other materials. BDF-Bismethine colorants are essentially different from BDF colorants in both their structure, electronics, and synthesis.

There are many compounds known which constitute a single lactone, lactam, or thio-lactone ring attached to an aromatic ring. Many of these are useful pharmaceutical intermediates. There are also many known compounds which constitute two lactones, thiolactones, or lactams attached to an aliphatic structure (J. Chem. Soc. 1957, p. 327). A few aromatic dilactones, dilactams, or dithiolactones are known. The aromatic dilactones, dilactams, and dithiolactones which are known to have been disclosed (Helv. Chim. Acta, 18, 1935, p. 613, Chem. Lett. 1983, p. 905, and J. Am. Chem. Soc. 66, 1944, p. 1541) are shown below. 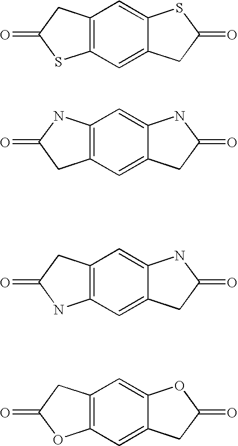
This class of compounds could be classified as bislactoarenes (BLA). It has been reported that some BLA compounds can exist in different tautomeric forms as exemplified below. (J. Chem. Soc. Transactions, 1922, 2640). 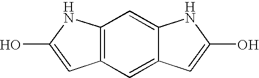
New synthetic methods for synthesizing novel aromatic dilactones, dilactams, and dithiolactones are desired. The aromatic ortho-Claisen rearrangement is a well know reaction in organic chemistry (Advanced Organic Chemistry, 4^{th} Ed. Jerry March editor p. 1136). A general scope of this reaction is shown below.

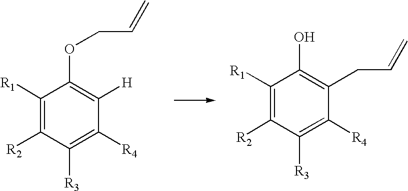

The aromatic ortho-Claisen rearrangement is versatile and in general could be applied to many aromatic allyloxy compounds. with a free C—H group adjacent on the aromatic ring. Two analogous reactions to the aromatic ortho-Claisen reaction are also known, the aromatic amino-Claisen and the aromatic thio-Claisen (Chem. Rev., 84, 3, 1984, p. 245 and p. 233) A general scope of these reactions is shown below.

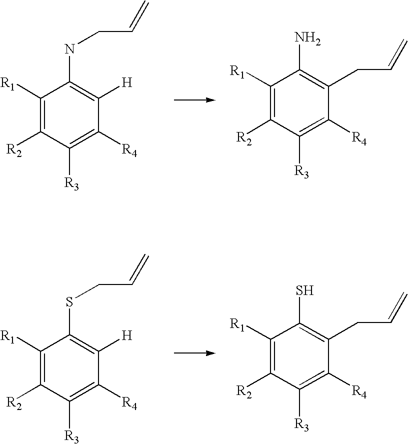

Sometimes it is useful to use a sulfide intermediate to facilitate the thio-Claisen. The ortho-amino-Claisen rearrangement is often catalyzed by Lewis Acids. These reactions are also versatile and apply to many aromatic allylamino or allythio (or allylsulfide or sulfone) compounds with an adjacent C—H group on the aromatic ring.

The Mukaiyama reaction is a known reaction in organic chemistry (March, p. 940). In its most common form, a silyl-enol ether or silyl ketene acetal is reacted with an aldehyde, ketone, or acetal in the presence of a Lewis-Acid catalyst. This catalyst is often TiCl₄, but many others are known.

Considerable efforts have been invested in developing alternative colorants, and methods for synthesizing BDF colorants. What is needed in the industry is one or more compounds having chromophores that can provide a wide color space with a single structure type. Compounds are needed that provide good thermal stability and greater color strength than known chromophores. Compounds are needed that can provide these advantages, which can be synthesized quickly and inexpensively. Furthermore, compounds also are needed that have the ability to decolorize when treated with certain reagents such as peroxy radicals or reducing agents, providing the ability to chemically decolorize an article. Such compounds may be useful in recycling and other applications.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention.

This invention provides examples of many different colorant compounds that exhibit high color strength, bright shades, and high thermal stability. These colorants may have found application as colorants for polyethylene terephthalate (“PET”) or other thermoplastics or thermosets. Potential end uses include disperse dyes, non-warping pigments, inks, pigments, solvent dyes, decolorizable colorants, dyes for electronic recording media and the like. These colorants provide the possibility to offer an entire color space with one class of chromophore.

The chromophores may be compatible with decolorizable colorants technology. The decolorization ability of such compounds is also very useful, and would make such a class of compounds ideally suited for decolorizing colorant technologies. Further, a novel synthetic strategy is provided herein for making a wide range of dilactones, dilactams, and dithiolactones.

The invention may provide a wide range of compositions of matter. First, it may involve the manufacture of bis-lactones from aromatic alcohols by a synthetic sequence:

1. Making a bis-allyoxy aromatic,

2. Performing an aromatic ortho-Claisen rearrangement,

3. Optionally protecting the free phenolic compound,

4. Oxidatively cleaving the allyl compound,

5. Optionally deprotecting the phenolic compound, and

6. Ring-closing the resulting compound to form a lactone.

Any or all of these steps may be combined into a single reaction. Further, the invention may be directed to the manufacture of lactams and thiolactones as well by the analogous amino-Claisen and thio-Claisen route using the analogous same sequence of steps. These novel aromatic dilactones, dilactams, and dithiolactones may be employed as pharmaceutical intermediates. Aromatic dilactones, aromatic dilactams, and aromatic dithiolactones can be grouped into a class known as Bislactoarenes.

The invention also may be directed to BDF bismethines made by condensing aldehydes (or other synthetic equivalent) to aromatic lactones. More generally, the invention may be directed to BLA bismethines made by condensing aldehydes (or other synthetic equivalent) to aromatic dilactones, dilactams and dithiolactones. The invention is also directed to the method of synthesis of BLA-bismethine compounds by using a silyl-enol ether intermediate as shown exemplified below. In the case of aromatic dilactams, a protecting group on the nitrogen may be beneficial.

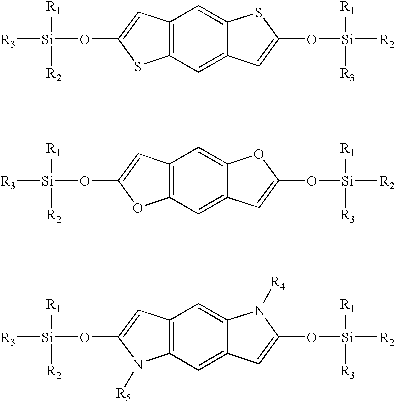

The Mukaiyama reaction may be employed in the practice of the invention. The invention includes as well methods for preparing several novel BLA bismethine colorants using the Mukaiyama aldol condensation. The Mukaiyama route is not the only way in which these compounds can be prepared (standard aldol condensation for example), but it provides a convenient route since the dilactone, dilactam, or dithiolactone is made relatively more soluble.

In one embodiment of the invention, the inventive BLA-Bismethine compound may exist in different tautomeric forms. A general example is shown below.

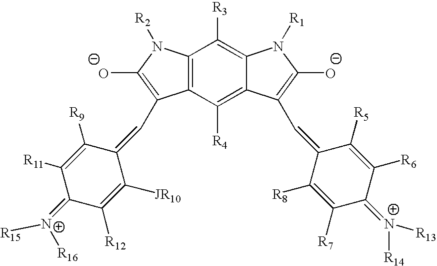

In one embodiment of the invention, a compound as below is disclosed: 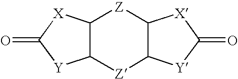
wherein, X, X′, Y, and Y′ are independently selected from the group consisting of: Nitrogen, Oxygen, Sulfur, and CH₂.

Furthermore, if X is Nitrogen, Oxygen, or Sulfur, then Y may be CH₂. If X′ is Nitrogen, Oxygen, or Sulfur, then Y′ may be CH₂.

If Y is Nitrogen, Oxygen, or Sulfur, then X may be CH₂. If Y′ is Nitrogen, Oxygen, or Sulfur, then X′ may be CH₂.

Z and Z′ constitute linking groups connecting the two lactones. This linking group may be a substituted or unsubstituted aromatic, substituted or unsubstituted heteroaromatic.

One embodiment of the invention a compound of the following structure is disclosed which is manufactured using as reactants an aldehyde (or other synthetic equivalent) condensed with the bislactone, bislactam, or bisthiolactone.

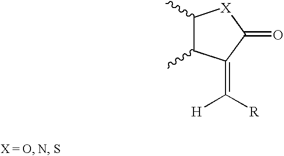

In the above compound, R may be essentially any aromatic, alkyl, aliphatic, heterocyclic, or conjugated group. This class of colorants covers a wide color space.

The invention in one aspect may comprise a compound of the structure: 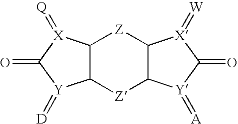
(a) wherein X, X′, Y, and Y′ are independently selected from the group consisting of: nitrogen, oxygen, sulfur, and carbon;
(b) wherein
   if X is nitrogen, oxygen, or sulfur, then Y is carbon; and
   if X′ is nitrogen, oxygen or sulfur, then Y′ is carbon; and
   if Y is nitrogen, oxygen or sulfur, then X is carbon, and
   if Y′ is nitrogen, oxygen or sulfur, then X′ is carbon; and
(c) wherein Z and Z′ taken together comprise a linking group, said linking group being selected from the following: substituted aromatic, unsubstituted aromatic, substituted heteromatic, unsubstituted heteroaromatic, substituted polyaromatic or unsubstituted polyaromatic and
(d) wherein Q, W, D, and A independently may or may not be present, further wherein a maximum of two of Q, W, D, and A may be present in said compound, and
(e) wherein
   if Q is present then D is not present; and
   if W is present then A is not present; and
   Q, W, D, and A always are attached to a carbon atom, and
   Q, W, D, and A can be the same or different and are independently selected, and if present, may have the general structure 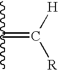
(f) wherein R may be selected from one or more of the following: C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, dialkylaminoaromatic, sulphonylamino, acyl, aryl, substituted aryl, heteroaryl, allyl, alkenyl, alkynyl, oxyalkylene, polyoxyalkylene, polyoxyalkylene substituted aromatic, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, substitiuted aromatics, aniline derivatives, 2,5-dimethoxyaniline derivatives, phenol derivatives, polyoxyalkylene substituted aniline derivatives, and polyoxyalkylene substituted phenol derivatives.

The compound of claim 16 where Z and Z′ taken together comprise a substituted or unsubstituted benzene ring. The compound as above may be provided in which Z and Z′ taken together may comprise a substituted or unsubstituted naphthalene ring. Z and Z′ taken together may comprise a substituted or unsubstituted anthraquinone ring. Z and Z′ taken together may comprise a substituted or unsubstituted anthracene ring.

Z and Z′ may constitute a substituted or unsubstituted heteroaromatic ring system. Further, in one option, at least one of X, X′, Y, or Y′ comprises an oxygen atom. In yet another embodiment, at least one of X, X′, Y, or Y′ may be a nitrogen atom. Further, at least one of X, X′, Y, or Y′ may be a sulfur atom. R may comprise a substituted aromatic.

A method of preparing a BLA compound (see further herein) also is disclosed where a silyl enol ether intermediate is employed in said method. A plastic article comprising at least in part the composition of the structure shown above may also be disclosed.

### SYNTHESIS EXAMPLES

### UV-Vis Spectroscopy

All UV-Vis spectra were collected using a Hewlett Packard 8452A diode array spectrophotometer. All samples were sequentially diluted in chloroform. Absorbance data was used to calculate molar absorptivities. Stama 5Q quartz spectrophotometer cells were used for UV-Vis studies.

### Synthesis of 2,3-diallyl-5,6-dimethyl hydroquinone

To a 250 mL round bottom flask, 5 g (36.1 mmol) of 2,3-dimethylhydroquinone, 6.3 mL (72.3 mmol) of allyl bromide, 10.1 g (72.3 mmol) of potassium carbonate, and 21.3 mL (289.5 mmol) of dry acetone were added. The solution was set to reflux overnight under a drying tube. It was next vacuum filtered to remove the potassium carbonate. An extraction was then performed on the filtrate with ethyl ether (100 mL), and the ether layer was collected. The ether layer was washed twice with 50 mL of 1 M sodium hydroxide to remove any phenolic excess. It was next washed twice with 20 mL of purified water, and the ether layer was collected. The ether layer was dried over sodium sulfate, and then it was vacuum filtered to remove the sodium sulfate. An orange-yellow solution remained in the bottom of the flask, Silica gel column chromatography was performed on the solid to obtain the pure intended product. The solid was dissolved in chloroform. The column was made of chloroform. The product was placed on the rotary evaporator and the solvent was removed to give diallyl 2,3-dimethyl hydroquinone ether as a thick oil. Thin Layer Chromatography (chloroform) of the product had an Rf value of 0.75.

To a 50 mL round bottom flask containing the dimethyl hydroquinone diallyl ether was added 15 mL of dodecane. The system was degassed with N₂ for five minutes. The solution was refluxed at 220° C. for 2¼ hours. The solution was stirred at room temperature overnight. At which time the solid was isolated by vacuum filtration, and the residue was washed with hexanes and air-dried. 2,3-Diallyl 5,6-dimethyl hydroquinone was isolated as a beige solid (3.92 g, 57% overall yield).

### Synthesis of 2,3-diallyl 1,4-dihydroxynapthalene

To a 250 mL round bottom flask, 2.68 g (16.7 mmol) of 1,4-dihydroxynapthalene, 2.9 mL (33.5 mmol) of allyl bromide, 4.61 g (33.5 mmol) of potassium carbonate, and 15.0 mL (133.9 mmol) of dry acetone were added. The solution was set to reflux overnight under a drying tube. It was next vacuum filtered to remove the potassium carbonate. An extraction was then performed on the filtrate with ethyl ether (100 mL), and the ether layer was collected. The ether layer was washed twice with 50 mL of 1 M sodium hydroxide to remove any phenolic excess. It was next washed twice with 25 mL of purified water, and the ether layer was collected. The ether layer was dried over sodium sulfate, and then it was vacuum filtered to remove the sodium sulfate. A brown oily residue remained in the bottom of the flask. Silica gel column chromatography was performed on the residue to obtain the pure product. The residue was dissolved in chloroform. The column was made of chloroform. The product was placed on the rotary evaporator and the solvent was removed. Thin Layer Chromatography was performed on the final product as an viscous clear oil. The chamber was filled with chloroform. The product had an Rf value of 0.78.

To a 50 mL round bottom flask containing the diallyl 1,4-dihydroxynapthalene ether was added 13 mL of dodecane. The system was degassed with N2 for five minutes. The solution was refluxed at 220° C. for 2¼ hours. The solution was stirred overnight at which time a solid had formed. The solid was isolated by vacuum filtration and the residue was washed with hexanes. 2,3-diallyl 1,4-dihydroxynapthalene was isolated as a fine, tan power (3.08 g, 77% yield). It was found to have a melting point between 132-138° C.

### Synthesis of 1,5-Bis(allyloxy)anthraquinone

To a 500 mL round-bottomed flask equipped with a stir bar was added 1,5-dihydroxyanthraquinone (anthrarufin) (1.00 g, 4.17 mmol) and 150 mL of acetone. The flask was then charged with powdered K₂CO₃ (7.00 g, 50.6 mmol) and allyl bromide (4 mL, 46.2 mmol). This mixture was heated at reflux for five days.

The hot mixture was then filtered with suction to remove residual solids, and the filtrate was concentrated by rotary evaporation. The resulting solid was then dissolved in 100 mL of CH₂Cl₂ and washed twice with 100 mL portions of water. The organic layer was dried (Na₂SO₄), filtered through phase separator paper, and concentrated using a rotary evaporator. Any remaining solvent was removed under high vacuum to give the desired product in 93% yield (1.23 g, 3.84 mmol). ¹H NMR (500 MHz, CDCl₃): 4.8 (d, 2H), 5.4 (d, 1H), 5.7 (d, 1H), 6.1 (q, 1H), 7.2 (d, 1H), 7.7 (t, 1H), 7.9 (d, 1H) ppm. IR (Diamond ATR): 3015, 2867, 1661, 1582, 1470, 1448, 1437, 1403, 1323, 1273, 1251, 1188, 1169, 1121, 1088, 1061, 1023, 984, 968, 936, 904, 849, 807, 765, 709, 617, 575 cm⁻¹. Elemental analysis: Calculated: C, 75.00%, H, 5.00%, O: 20.00%. Found: C: 74.61%, H, 5.05%.

### 1,5-Dihydroxy-2,6-diallylanthraquinone

A 50 mL round-bottomed flask was charged with 1,5-bis(allyloxy)-anthraquinone (200 mg, 0.62 mmol) and then sealed with a rubber septum stopper. The flask was fitted with a gas inlet and outlet and then flushed with nitrogen for ten minutes. The vessel was heated with constant agitation under a steady flow of nitrogen in a pre-heated 220° C. mineral oil bath for 30 minutes. The vessel was then allowed to cool to room temperature under a positive pressure of nitrogen. The crude Claisen product (contaminated with deallylated 1,5-dihydroxyanthraquinone) was purified by column chromatography (silica gel, hexane/chloroform 9:1) to give the desired product in 40% yield (50 mg, 0.25 mmol). ¹H NMR (500 MHz, CDCl₃): 3.5 (d, 2H), 5.2 (d, 2H), 6.0 (q, 1H), 7.6 (d, 1H), 7.8 (d, 1H) ppm. IR (Diamond ATR): 2918, 2850, 1742, 1718, 1633, 1596, 1580, 1476, 1422, 1367, 1324, 1291, 1243, 1205, 1066, 1017, 984, 970, 913, 851, 814, 793, 773, 729, 617 cm⁻¹. Elemental analysis: Calculated: C, 74.98%, H: 5.04%, O: 19.98%. Found: C, 75.12%, H, 6.44%.

### Synthesis of 1,2-diallyoxybenzene

To a 250 mL round bottom flask, 5.0 g (61.7 mmol) of catechol, 14.9 mL (123.4 mmol) of allyl bromide, 17.0 g (33.5 mmol) of potassium carbonate, and 28.7 mL (493.6 mmol) of dry acetone were added. The solution was set to reflux overnight under a drying tube. It was next vacuum filtered to remove the potassium carbonate. An extraction was then performed on the filtrate with ethyl ether (100 mL), and the ether layer was collected. The ether layer was washed twice with 50 mL of 1 M sodium hydroxide to remove any phenolic excess. It was next washed twice with 25 mL of purified water, and the ether layer was collected. The ether layer was dried over sodium sulfate, and then it was vacuum filtered to remove the sodium sulfate. A yellow liquid remained in the bottom of the flask. Thin Layer Chromatography was performed on the final product. The chamber was filled with chloroform. The product had an Rf value of 0.64.

### Synthesis of 2,5-, and 2,3-diallyl hydroquinone

To a 250 mL round bottom flask, 10 g (90.8 mmol) of hydroquinone, 50 mL (681.0 mmol) of dry acetone, 15.7 mL (181.4 mmol) of allyl bromide, and 25.5 g (184.5 mmol) of potassium carbonate were added. The solution was set to reflux overnight under a drying tube. It was next vacuum filtered to remove the potassium carbonate. An extraction was then performed on the filtrate with ethyl ether (100 mL), and the ether layer was collected. The ether layer was washed twice with 50 mL of 1 M sodium hydroxide to remove any phenolic excess. It was next washed twice with 25 mL of purified water, and the ether layer was collected. The ether layer was dried over sodium sulfate, and then it was vacuum filtered to remove the sodium sulfate. The solution was placed on the rotary evaporator and the ether was removed. An orange-yellow solution remained in the bottom of the flask. To the flask was added 50 mL of 95% ethanol. The solution was put into the freezer and crystals formed. (7 g, 10.5 mmol, 41% yield) TLC (20% Ethyl acetate/hexane) Rf=0.667

To a 50 mL round bottom flask, 2 g (105 mmol) of hydroquinone diallyl ether was added to 13 mL (57.2 mmol) of dodecane. The system was degassed with N₂ for five minutes. The solution was refluxed at 220° C. for 2¼ hours. The solution was vacuum filtered. The solution and the solid were washed with petroleum ether. The solid was collected. Silica gel column chromatography was performed on the solid to obtain the two separate isomers. The solid was dissolved in ethyl acetate and methylene chloride. The column was made of silica in a solution of 10% ethyl acetate, 10% methylene chloride, and 80% hexanes. The products obtained were placed on the rotary evaporator and the solvent was removed.

2,5-diallylhydroquinone was isolated as a white solid, which was recrystallized from ethyl acetate/hexane. (0.7 g, 3.68 mmol, 35% yield) TLC (20% ethyl acetate/hexane) Rf=0.333 2,3-diallylhydroquinone was isolated as a white fibrous solid, which was recrystallized from ethyl acetate/hexane. (0.7 g, 3.68 mmol, 35% yield) TLC (20% ethyl acetate/hexane) Rf=0.167

### Synthesis of 2,5-diallylhydroquinone diacetate

In a 250 mL round bottom, 0.54 g (2.84 mmol) of the diol was dissolved in 5 mL (78.10 mmol) dry methylene chloride, 1 mL (12.48 mmol) dry pyridine, and 4 mL (42.39 mmol) acetic anhydride. The solution was stirred to two days and its completion was confirmed by a thin layer chromatography. The solution was worked-up by adding 50 mL chloroform and 20 mL water. The chloroform layer was collected and dried over sodium sulfate. The chloroform layer was then pumped down on the rotary evaporator to leave an oil residue with a smell of pyridine and acetic anhydride. The solution was placed on the Kugelrohr at 0.05 mmHg at 70° C. to form a golden oil. Upon returning to room temperature, the oil solidified. (0.670 g, 2.45 mmol, 86% yield) TLC (20% ethyl acetate/hexane) Rf=0.611

### Synthesis of 2,5-dihydroxybenzene-1,4-diacetic acid diacetate

In a 250 mL two-necked round bottom flask, 0.670 g (2.45 mmol) of the protected hydroquinone dissolved in 10 mL (156.2 mmol) dry methylene chloride and 10 mL (346.4 mmol) dry methanol. The system was flushed with oxygen gas for 5 minutes. While flushing the system, the solution was cooled to between −15° C. and −25° C. in a water/ethanol/liquid nitrogen gas. Ozone was then flushed through for 20 minutes. The settings on the ozonator were 0.58 amps, 3 psi, and 8 mL/min. The solution changed from golden to colorless and was stirred for 1 hour to return to room temperature. The system was flushed with nitrogen for 20 minutes to get rid of any excess ozone and then vacuum aspirated for 5 minutes. The solution was placed on the rotary evaporator and pumped to dryness. White crystals formed in the round bottom flask. 6 mL of formic acid and 3 mL of hydrogen peroxide were added to the flask and stirred overnight. Completion was confirmed by thin layer chromatography. The solution was worked-up by extraction with 100 mL ethyl acetate and 50 mL water. The ethyl acetate layer was collected and dried over sodium sulfate. It was then pumped down on the rotary evaporator to dryness. The remaining golden yellow oil dried overnight in the fume hood. (0.401 g, 1.29 mmol, 53%)

### Synthesis of 2,5-dihydroxybenzene-1,4-diacetic acid

The protected diacid (0.09 g, 0.29 mmol) was dissolved in 3 mL 2% sulfuric acid and stirred over the weekend. Extraction was performed 3 times with 15 mL ethyl acetate and 5 mL water. The ethyl acetate layer was collected and the solvent pumped off on the rotary evaporator. Brownish solid residue remained after drying in the fume hood. (0.045 g, 0.23 mmol, 78% yield) TLC (20% methanol/chloroform) R_{f}=0.190

### Synthesis of 2,5-dihydroxybenze-1,4-diacetic acid di-gamma-lactone

The BDF dilactone was formed by treatment with acetic anhydride in toluene following the procedure of Wood et al. J. Am. Chem. Soc. 66, 1944, p. 1541.

### p-Dibutylaminobenzaldehyde BDF bismethine

An oven-dried 50 mL round-bottomed flask equipped with a stir bar was charged with 2,5-dihydroxybenzenediacetic acid di-gamma-lactone (0.95 g, 5.00 mmol) and sealed with a septum stopper. Purified THF (10 mL) was then added through the septum stopper by syringe. The flask was fitted with a nitrogen inlet and outlet (connected to a bubbler) and cooled to −78° C. (isopropyl alcohol/CO₂) under a positive flow of nitrogen. To the cooled stirring slurry was then added by syringe 5 mL of a 2M LDA solution in THF. The solution was allowed to stir for ten minutes.

Chlorotrimethylsilane (2.8 mL, 22.1 mmol) was added to the lithium dienolate solution by syringe. The resulting mixture was stirred for two minutes and then removed from the cooling bath and the stir plate. The mixture was allowed to settle under a static nitrogen atmosphere (i.e. no positive flow) for one hour.

Meanwhile, an oven-dried 100 mL round-bottomed flask equipped with a stir bar was charged with p-dibutylaminobenzaldehyde (1.8 mL, 7.54 mmol) and then sealed with a septum stopper. Purified methylene chloride (20 mL) was added and the flask was cooled to −78° C. (isopropyl alcohol/CO₂) under a positive flow of nitrogen. A 1M titanium tetrachloride solution in methylene chloride (11 mL) was added by syringe to the p-dibutylaminobenzaldehyde solution, which was allowed to stir for two minutes.

To the stirring p-dibutylaminobenzaldehyde/TiCl₄ solution was added the supernatant liquid from the settled disilyl ketene acetal mixture. The final mixture was allowed to stir for one hour while warming to room temperature. After reaching room temperature, the septum stopper was removed, and the flask was charged with 50 mL of water. The flask was resealed, and the mixture was stirred at room temperature overnight.

The reaction mixture was allowed to separate in a separatory funnel for about three hours. The organic layer was filtered with suction to remove solids and then the filtrate was diluted with 100 mL of methylene chloride. The organic layer was dried (Na₂SO₄), filtered through phase separator paper, and concentrated using a rotary evaporator. Any residual solvent was evaporated under high vacuum.

The crude product was then purified by column chromatography (silica gel) using a hexane/chloroform solvent system (2:1) until the unreacted aldehyde had eluted (followed by TLC). The column was then eluted using 10% methanol in chloroform until the desired product came off in the eluent. The resulting red dye was isolated in 10.3% yield (320 mg, 0.52 mmol). ¹H NMR (500 MHz, DMSO-d₆): 0.9 (t, 3H), 1.3 (q, 2H), 1.5 (q, 2H), 3.5 (t, 2H), 6.8 (m, 2H), 7.6 (d, 1H), 7.7 (m, 2H), 8.4 (d, 1H) ppm. IR (Diamond ATR): 2955, 1741, 1558, 1516, 1445, 1364, 1180, 1069, 1029, 986, 816 cm⁻¹. UV-Vis (CHCl₃): Lambdaₘₐₓ=574 nm, Epsilon=60,431. Elemental analysis: Calculated: C, 77.40%, H, 7.80%, N: 4.50%, O: 10.30%. Found: C, 77.14%, H, 7.76%, N, 4.53%.

### Synthesis of 2,3-dihydroxybenzene-1,4-diacetic acid di-lactone

2,3-dihydroxybenzene-1,4-diacetic acid was prepared from 2,3-diallyloxybenzene following the same procedures as shown above for the 2,5-dihydroxybenzene-1,4-diacetic acid. The 2,3-dihydroxybenzene-1,4-diacetic acid was ring-closed to form the dilatone following the procedures of Wood et. al. The dilactone was purified via column chromatography (chloroform followed by 2% MeOH/chloroform). The product was a white crystalline material. ¹H NMR (500 MHz, DMSO-d₆): 3.8 (s, 4H), 7.05 (s, 2H). IR (KBr): 1820 cm⁻¹ (lactone).

### Synthesis of the BDF-Bismethine compound based 2,3-dihydroxy-1,4-benzenediacetic acid di-lactone (catechol based dilactone)

2,3-dihydroxy-1,4-benzenediacetic acid di-lactone was condensed with alkoxylated (16EO 10 PO) para-formyl aniline (prepared according to U.S. Pat. No. 4,594,454 to Moore et al.) following the procedure given in example 1 of U.S. Pat. No. 6,492,533 to Connor et. al. A red dye was obtained with a Lambdamax=around 540 nm. The purified colorant was a liquid at room temperature.

### Synthesis of bis(1,5-allyloxy)-naphthalene (Allylation of 1,5-dihydroxynaphthalene)

In a 1000 mL round-bottomed flask equipped with a stir bar was added 1,5-dihydroxynaphthalene (26.8 g, 167 mmol). The flask was then charged with 150 mL of acetone and 150 mL of purified THF. Stirring was begun to ensure adequate dissolution of the 1,5-dihydroxynaphthalene. To the stirring solution was added granular potassium carbonate (46.1 g, 335 mmol, 2.4 equivalents) followed by allyl bromide (29 mL, 335 mmol, 2.4 equivalents). The resulting mixture was heated overnight at reflux.

After cooling to room temperature, the mixture was diluted with 500 mL of ethyl ether and washed twice with 500 mL portions of a 1N sodium hydroxide solution, followed by a wash with 500 mL of water. The organic layer was then collected and dried over sodium sulfate for several hours. The mixture was filtered through phase separator paper and concentrated using a rotary evaporator. Any remaining solvent was then removed under high vacuum. The desired product was isolated as a brown solid in 92% yield (36.95 g, 154 mmol).

An analytical sample was prepared by washing 1.00 g of the product with two 20 mL portions of hexane. The cleaned product was freed of most of the solvent by suction filtration. The remaining hexane was removed in vacuo to give 150 mg of clean product. ¹H NMR (CDCl₃, 500 MHz): 4.7 (d, 2H), 5.3 (d, 1H), 5.5 (d, 1H), 6.3 (m, 1H), 6.8 (d, 1H), 7.4 (t, 1H), 7.9 (d, 1H) ppm. IR (Diamond ATR): 3064, 3020, 2919, 1592, 1509, 1405, 1267, 1036, 919, 773 cm⁻¹. Elemental analysis: Calculated: C, 79.96%, H, 6.72%, O: 13.32%. Found: C: 80.22%, H, 6.84%.

### Synthesis of 1,5-Dihydroxy-2,6-diallyinaphthalene

A 200 mL round-bottomed flask was charged with 1,5-bis(allyloxy)-naphthalene (5.4 g, 22.5 mmol) and then was sealed with a septum stopper. The flask was fitted with a gas inlet and outlet and then flushed with nitrogen for ten minutes. The vessel was heated with constant agitation under a steady flow of nitrogen in a pre-heated 190° C. mineral oil bath for ten minutes. The vessel was then allowed to cool to room temperature under a positive pressure of nitrogen. The rearranged Claisen product was isolated in 100% yield (5.4 g, 22.5 mmol).

An analytical sample was prepared by pulverizing 975 mg of the Claisen product and washing it with two 15 mL portions of hexane. The cleaned Claisen product was isolated by filtration with suction. The remaining hexane was removed in vacuo to give 815 mg of purified Claisen product. ¹H NMR (CDCl₃, 500 MHz): 3.6 (d, 2H), 5.2 (d, 2H), 5.5 (s, 1H), 6.1 (m, 1H), 7.2 (d, 1H), 7.8 (d, 1H) ppm. IR (Diamond ATR): 3333, 3079, 2978, 2919, 2861, 1592, 1509, 1406, 1380, 1362, 1267, 1245, 1210, 1037, 989, 913, 870, 775 cm⁻¹. Elemental analysis: Calculated: C, 79.96%, H, 6.72%, O: 13.32%. Found: C, 80.07%, H: 6.79%.

### Synthesis of 1,5-Dibenzyloxy-2,6-diallyinaphthalene

In a 1000 mL round-bottomed flask equipped with a stir bar, 1,5-dihydroxy-2,6-diallylnapthalene (16.9 g, 70.1 mmol) was dissolved in a mixture of 100 mL of acetone and 100 mL of purified THF. To this stirred mixture was added granular potassium carbonate (21.0 g, 154 mmol, 2.2 equivalents) and benzyl bromide (18.3 mL, 154 mmol, 2.2 equivalents). The resulting mixture was stirred overnight at reflux.

After cooling to room temperature the mixture was diluted with 200 mL of ethyl ether and was washed twice with 200 mL portions of aqueous 1N sodium hydroxide, followed by two washes with 200 mL portions of water. The organic layer was then retrieved and dried over sodium sulfate, filtered through phase separator paper, and concentrated using a rotary evaporator. The remaining solvent and some remaining benzyl bromide were then removed in vacuo. The benzyl bromide-contaminated product was removed from the round-bottomed flask and spread in a thin layer on a large watch glass and was left to dry in the hood for several days. The desired product was isolated as a dark orange solid in a yield of 23.9 g (56.8 mmol, 81%).

An analytical sample was prepared by washing 1.00 g of the crude product with three 20 mL portions of petroleum ether. The cleaned product was then isolated by filtration with suction. The remaining petroleum ether was evaporated under high vacuum to give 250 mg of purified product. ¹H NMRa (CDCl₃, 500 MHz): 3.6 (d, 2H), 5.1 (m, 2H), 6.1 (m, 1H), 7.4 (m, 5H), 7.6 (d, 1H), 7.9 (d, 1H) ppm. IR (Diamond ATR): 3068, 3038, 2969, 2901, 1636, 1600, 1454, 1438, 1403, 1364, 1335, 1279, 1243, 1173, 1081, 1034, 1027, 995, 974, 908, 881, 833, 817, 740, 698 cm⁻¹. Elemental analysis: Calculated: C, 85.66%, H, 6.72%, O: 7.62%. Found: C, 85.46%, H, 6.70%.

### Synthesis of 1,5-Dibenzyloxynaphthalene-2,6-diacetaldehyde

To a two-neck 250 mL round-bottomed flask equipped with a stir bar was added 1,5-dibenzyloxy-2,6-diallylnaphthalene (2.0 g, 4.76 mmol). The solid was dissolved in 30 mL of acetone. Oxygen was bubbled through the solution for five minutes while the flask was cooled to −78° C. in an isopropyl alcohol/dry ice bath. After the five-minute flush with oxygen, ozone (flow rate: 6 mL/min, pressure: 3 psi, rheostat: 0.6 A) was bubbled through the solution for 15 minutes followed by nitrogen for one hour. (Note: Careful monitoring of the reaction time with ozone is essential to ensure that the reaction proceeds correctly. Insufficient reaction time gives a mixture of product and starting material, while longer reactions times result in degradation of the aromatic core of the molecule.) As nitrogen was passed through the solution the reaction vessel was allowed to warm to 0° C. in an ice bath.

The solution was diluted with 30 mL of acetone and then treated with zinc dust (7.0 g) added in small increments and 75% acetic acid (8 mL), similarly added incrementally. The resulting mixture was allowed to stir for another 30 minutes while warming to room temperature.

The zinc dust was then removed by filtration with suction, and the acetone filtrate was concentrated using a rotary evaporator. The resulting yellow oil was then redissolved in 100 mL of methylene chloride. That solution was washed twice with 100 mL of aqueous 1N sodium bicarbonate. The organic layer was isolated, dried (Na₂SO₄), filtered through phase separator paper, and concentrated using a rotary evaporator. Any remaining solvent was removed in vacuo to give a sticky yellow solid in 81% yield (1.64 g, 3.9 mmol). ¹H NMR (CDCl₃, 500 MHz): 3.8 (s, 2H), 5.0 (s, 2H), 7.4 (m, 5H), 7.5 (d, 1H), 8.0 (d, 1H), 9.8 (s, 1H) ppm. IR: 3066, 3032, 2873, 1720, 1602, 1497, 1454, 1401, 1362, 1337, 1244, 1171, 1105, 1079, 1050, 1002, 918, 822, 735, 697 cm⁻¹. Elemental analysis: Calculated: C, 79.21%, H, 5.71%, O: 15.08%. Found: C, 75.69%, H: 5.71%. (Note: Found values are consistent with calculated values for the desired molecule with one water of hydration: C, 75.99%, H, 5.93%, O: 18.08%.)

### Synthesis of 1,5-Dibenzyloxynaphthalene-2,6-diacetic acid

The 1,5-dibenzyloxynaphthalene-2,6-diacetaldehyde (1.58 g, 3.72 mmol) was dissolved in 30 ml of acetone in a 200 mL round-bottomed flask equipped with a stir bar. The stirring solution was titrated with Jones reagent (CrO₃/H₂O/H₂SO₄) until the mixture became a persistent dark green/black (ca. 6 mL added dropwise). After stirring for thirty minutes at room temperature, the mixture was filtered through a filter paper cone to remove chromium salts. The acetone solution was then concentrated using a rotary evaporator. Water (50 mL) was added to the resulting oily paste. The water layer was then extracted with three 50 mL portions of ethyl acetate. The organic layers were combined, dried (Na₂SO₄), filtered through phase separator paper and concentrated using a rotary evaporator. The remaining solvent was removed in vacuo to give a light yellow solid in 75% yield (1.27 g, 2.79 mmol).

An analytical sample was prepared by washing 616 mg of crude product with two 10 mL portions of methylene chloride followed by two 10 mL portions of hexane. The purified product was isolated by filtration with suction. Any remaining solvent contamination was removed in vacuo to give 320 mg of the desired product. ¹H NMR (DMSO-d₆, 500 MHz): 3.7 (s, 2H), 5.0 (s, 2H), 7.4 (m, 5H), 7.6 (d, 1H), 7.9 (d, 1H), 12.4 (broad s, 1H) ppm. IR (Diamond ATR): 3065, 3030, 2935, 1705, 1602, 1499, 1455, 1402, 1359, 1327, 1234, 1212, 1182, 1044, 1029, 973, 914, 893, 820, 769, 748, 696, 676, 624 cm⁻¹. Elemental analysis: Calculated: C, 73.66%, H, 5.31%, O: 21.03%. Found: C, 70.31%, H: 5.11%. (Note: The experimental values found were consistent with calculated values for the desired compound with one water of hydration: C, 70.86%, H: 5.53%, O: 23.61%.)

### Synthesis of 1,5-Dihydroxynaphthalene-2,6-diacetic acid

The 1,5-dibenzyloxynapthalene-2,6-diacetic acid (1.19 g, 2.61 mmol) was taken up in 100 mL of chilled methanol in a 500 mL round-bottomed flask. Catalyst (10% Pd/C) was carefully added to the cold methanolic solution. (Note: The methanol was chilled in order to prevent the catalyst from combusting when added to the flask.) The flask was then connected to an atmospheric hydrogenation apparatus and allowed to stir overnight under an atmosphere of hydrogen gas.

On the next day, the solution was filtered through a celite bed to remove catalyst and concentrated using a rotary evaporator. The remaining methanol was removed in vacuo to give the desired deprotected product in 97% yield (700 mg, 2.53 mmol).

An analytical sample was prepared by washing 100 mg of the crude product with three 10 mL portions of ethyl ether. The solid was then isolated by filtration with suction. Any remaining solvent was removed under high vacuum to give 95 mg of the desired product. ¹H NMR (500 MHz, DMSO-d₆): 3.7 (s, 2H), 7.2 (d, 1H), 7.6 (d, 1H) ppm. IR: 3292, 3016, 2650, 2593, 1675, 1602, 1447, 1421, 1399, 1383, 1349, 1297, 1262, 1240, 1208, 1163, 922, 893, 693 cm⁻¹. Elemental analysis: Calculated: C, 60.86%, H, 4.39%, O: 34.75%. Found: C: 59.69%, H, 4.49%.

### Synthesis of 1,5-Dihydroxynaphthalene-2,6-diacetic acid di-γ-lactone

A 250 mL round-bottomed flask was charged with the 1,5-dihydroxynaphthalene-2,6-diacetic acid (500 mg, 1.81 mmol) along with acetic anhydride (4.25 mL, 45 mmol) and 130 mL of toluene. The insoluble diacid was then refluxed with the acetic anhydride in toluene for two days. After two days, most of the solid had dissolved.

The hot solution was then filtered with suction to remove residual solids and was concentrated using a rotary evaporator. The remaining toluene and acetic anhydride was removed in vacuo to give the desired product in crude form. The crude dilactone was then washed three times with 20 mL of ethyl ether. The resulting brown solid was boiled in 50 mL of methanol and isolated by filtration with suction. The dilactone was dried of any remaining methanol in vacuo to give the desired product in 80% yield (350 mg, 1.46 mmol). ¹H NMR (500 MHz, DMSO-d₆): 4.2 (s, 2H), 7.6 (d, 1H), 7.8 (d, 1H) ppm. IR (Diamond ATR): 2922, 1781, 1625, 1532, 1386, 1298, 1240, 1183, 1115, 997, 929, 813, 733, 680, 668, 591, 526, 531 cm⁻¹. Elemental analysis: Calculated: C, 69.99%, H, 3.36%, O: 26.65%. Found: C, 65.07%, H, 4.10%. (Note: the experimental values found are consistent with the calculated values for the desired molecule with one water of hydration: C, 65.13%, 3.91%, O: 30.96%.)

### Synthesis of p-Dibutylaminobenzaldehyde Naphthalene difuranone Bismethine

A 50 mL round-bottomed flask equipped with a stir bar was charged with 1,5-dihydroxynaphthalene-2,6-diacetic acid di-lactone (426 mg, 1.77 mmol) and sealed with a septum stopper. Purified THF (10 mL) was then added through the septum stopper by syringe. The flask was fitted with a nitrogen inlet and outlet (connected to a bubbler) and cooled to 0° C. on an ice bath under a positive flow of nitrogen. To the cooled stirring slurry was then added by syringe 2 mL of a 2M LDA solution in THF. The solution was allowed to stir for ten minutes.

Chlorotrimethylsilane (1.0 mL, 7.9 mmol) was added to the lithium dienolate solution by syringe. The resulting mixture was stirred for two minutes and then removed from the cooling bath and the stir plate. The mixture was allowed to settle under a static nitrogen atmosphere (i.e. no positive flow) for one hour.

Meanwhile, another 50 mL round-bottomed flask equipped with a stir bar was charged with p-dibutylaminobenzaldehyde (890 L, 3.72 mmol) and then sealed with a septum stopper. Purified methylene chloride (10 mL) was then added, and the flask was cooled to 0° C. (ice bath) under a positive flow of nitrogen. A 1M titanium tetrachloride solution in methylene chloride (4 mL) was added by syringe to the p-dibutylaminobenzaldehyde solution, and the mixture was allowed to stir for two minutes.

To the stirring p-dibutylaminobenzaldehyde/TiCl₄ solution was added the supernatant liquid from the settled disilyl ketene acetal mixture prepared from the dilactone. The final mixture was allowed to stir overnight at room temperature.

The reaction mixture was concentrated using a rotary evaporator. The crude reaction mixture was then washed with several 20 mL portions of hexane. The solid phase was collected by filtration with suction.

This crude product was then purified by column chromatography (silica gel) using a chloroform/hexane solvent system (2:1) until the unreacted aldehyde had eluted (followed by TLC). The column was then eluted using pure methanol until the desired product was observed in the eluent. The resulting red dye was isolated in 13.5% yield (160 mg, 0.24 mmol). ¹H NMR (DMSO-d₆): 1.0 (t, 3H), 1.4 (q, 2H), 2.7 (q, 2H), 3.4 (t, 2H), 6.7 (d, 2H), 7.7 (d, 1H), 7.8 (d, 2H), 8.1 (d, 1H), 8.4 (d, 1H) ppm. IR (Diamond ATR): 2955, 2929, 2870, 1768, 1745, 1559, 1515, 1462, 1395, 1357, 1322, 1289, 1223, 1188, 1166, 1111, 1079, 1028, 1001, 947, 926, 908, 807, 727, 697, 667 ppm. UV-Vis (CHCl₃): λₘₐₓ=556 nm, ε=72799. Elemental analysis: Calculated: C, 78.76%, H, 7.53%, N, 4.18%, O: 9.53%. Found: C, 75.45%, H, 7.45%, N, 3.78%. (Note: The experimental values found are consistent with calculated values for the desired structure with two waters of hydration: C, 74.74%, H, 7.71%, N, 3.96%, O: 13.57%.)

### Synthesis of N,N-dibenzyl-5,7-dihydro-1H,3H-pyrrolo[3,2-f]indole-2,6-dione

(4,6-diamino-m-phenylene)-di-acetic acid diethyl ester (7.54 g) (prepared according to Helv. Chim. Acta 18, 1935, 613-620), benzaldehyde (19.01 g), and ethanol (50 mL) were heated at 75° C. for 1 h, cooled, and filtered to yield 9.18 g of the diimine. The diimine (8.03 g) was dissolved in anhydrous THF (65 mL) and placed in a water bath. Glacial acetic acid (9.6 g) was added to the THF solution. Sodium cyanoborohydride (2,4 g) was dissolved in THF (35 mL) and added slowly to the diimine/THF/acetic acid solution. The mixture was stirred at room temperature for 2 h. Water (10 mL) was added and the mixture stirred 10 minutes. The solution was neutralized with sodium carbonate, stirred 1 hour, and extracted with chloroform. The chloroform solution was washed twice with water and dried over sodium sulfate and the solvent removed under reduced pressure to yield the N,N-dibenzyl protected 4,6-diamino-m-phenylene)-di-acetic acid diethyl ester. The N,N-dibenzyl protected 4,6-diamino-m-phenylene)-di-acetic acid diethyl ester was taken up in toluene (150 mL) and heated to reflux for 1 h in the presence of paratoluenesulphonic acid (0.2 g), cooled in ice, and filtered to yield pure N,N-dibenzyl-5,7-dihydro-1H,3H-pyrrolo[3,2-f]indole-2,6-dione (4.87 g).

### Synthesis of BLA-Bismethine from N,N-dibenzyl-5,7-dihydro-1H,3H-pyrrolo[3,2-f]indole-2,6-dione and dibutylaminobenzaldehyde

N,N-dibenzyl-5,7-dihydro-1H,3H-pyrrolo[3,2-f]indole-2,6-dione (0.25 g), 4-dibutylaminobenzaldehyde (0.45 g), sodium hydroxide (anhydrous) (0.05 g), and 1,4-dioxane (20 mL) were heated at reflux for 4 hours under a nitrogen atmosphere. The solvent was removed under reduced pressure, the orange solid taken up in methylene chloride, and washed with water, and dried over Na₂SO₄. The solvent was removed under reduced pressure to afford the orange BLA-bismethine colorant. The colorant could be purified by column chromatography to give an intensely colored orange solid with a lambda max around 460 nm.

Method for the Preparation of BLA-Bismethine Compounds

BDF bismethine colorants were generated by first converting the appropriate dilactone, dilactam, or dithiolactone to the corresponding disilyl ketene acetal by treatment with LDA followed by chlorotrimethylsilane at −78° C. After the disilyl ketene acetal was prepared in situ, it was treated with the appropriate aromatic aldehyde in the presence of titanium tetrachloride to afford the Mukaiyama aldol condensation product.

Thermoplastic Compositions Containing BLA-Bismethine Colorants

The condensation product of N,N-dibenzyl-5,7-dihydro-1H,3H-pyrrolo[3,2-f]indole-2,6-dione and dibutylaminobenzaldehyde (3 mg) was added to 3 g of PET-G thermoplastic polymer at 250° C. The mixture was well mixed in a small heating well for 2 minutes. Compression molded press-out parts were made by pressing the molten polymer between metal plates. The resulting plastic article was colored a deep orange color. The process was repeated except that 0.8 IV polyethylene terephthalate was used and the colorant was mixed with the polymer at 300° C. A deeply orange colored plastic part was produced.

Definitions

1. “Bislactoarene (BLA)” refers to a molecule consisting of an aromatic ring system to which two lactam, lactone, or thiolactones are attached.

2. “Lacto” collectively refers to and includes: lactone, lactam, and thiolactone.

3. “BLA-Bismethine” refers to a BLA molecule to which two aldehyde (or equivalent synthon) equivalents have been condensed to form a bismethine moiety. Specifically, a double bond attaches a carbon atom to the carbon of the lacto ring of the BLA molecule.

4. “Benzene-centered” refers to organic structures wherein the two dilactones, dilactams, or dithiolactones are attached to a centrally located benzene ring or substituted benzene ring.

5. “Lactam” refers to a five-membered ring containing an amide functional group as part of the ring. The lactam is bound to an aromatic ring structure. R₁ and R₂ shown below represent carbons that are part of the attached single or multiple aromatic ring structure. 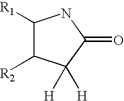
6. “Thiolactone” refers to a five-membered ring containing a thioester functional group as part of the ring. The thiolactone is bound to an aromatic ring structure. R₁ and R₂ shown below represent carbons of an attached single or multiple aromatic ring structure 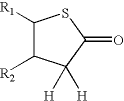
7. “Naphthalene-centered” refers to organic structures wherein the two dilactones, dilactams, or dithiolactones are attached to a centrally positioned naphthalene ring system.
8. “Lactone” refers to a five-membered ring containing an ester functional group as part of the ring. The lactone may be bound to an aromatic ring structure. R₁ and R₂ in the illustration below represent carbons which are part of an attached single or multiple aromatic ring structure. 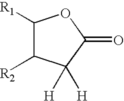
9. “Anthraquinone-centered” refers to organic structures where two dilactones, dilactams, or dithiolactones are attached to a centrally located anthraquinone ring system.
10. “Anthracene-centered” refers to organic structures wherein two dilactones, dilactams, or dithiolactones are attached to a centrally positioned anthracene ring system.
11. “Hetero-aromatic centered” refers to organic structures wherein two dilactones, dilactams, or dithiolactones are attached to a central aromatic ring system, wherin the ring system contains atoms other than carbon as components of ring structure or ring backbone.

### Applications of Compounds

Compositions comprising such compounds of are also encompassed within this invention. The compositions may include as well coloring agents, ultraviolet absorbers, light stabilizers, bluing agents, anti-oxidants, clarifiers, nucleating agents, or mixtures thereof, as liquids or as pellets for further introduction within desired molten thermoplastic or thermoset formulations (or precursor formulations). Methods of making such compositions, particularly thermoplastics, comprising such compounds of are also contemplated within this invention.

The term “thermoplastic” is intended to encompass any synthetic polymeric material that exhibits a modification in physical state from solid to liquid upon exposure to sufficiently high temperatures. Most notable of the thermoplastic types of materials are polyolefins (i.e., polypropylene, polyethylene, and the like), polyester (i.e., polyethylene terephthalate, polybutylene terephthalate, polytrimethylene terephthalate, and the like), polyamides (i.e., nylon-1,1, nylon-1,2, nylon-6 or nylon-6,6), polystyrenes, polycarbonates, polyvinyl halides (i.e., polyvinyl chloride and polyvinyl difluoride, as merely examples). Thermoplastics that are readily employed in the practice of the invention include polyesters and PET (polyethylene terephthalate).

Such thermoplastic articles may include bottles, storage containers, sheets, films, fibers, plaques, hoses, tubes, syringes. Included are polyester, polystyrene and other like resinous materials in sheet form which are present within windows for strength and resiliency functions. In such an instance, the inventive colorant compounds provide or contribute to excellent colorations to such thermoplastic articles for decorative, aesthetic or protective purposes. The possible uses for such a low-migratory, thermally stable colorant for such items as thermoplastics (particularly polyesters such as transparent polyethylene terephthalate) are many. Other possible end-uses include use of such compounds within solvent systems, printing inks, within and on textiles (either on or within textiles, fibers, or fabrics), within display devices such as liquid crystal displays, and the like.

The inventive colorant compounds may be added in any amount to such thermoplastics as is needed to provide beneficial results. The amount may be between about 0.00001 ppm to about 25,000 ppm per total amount of resin; more preferably from about 0.001 and about 15,000 ppm; in other applications may be between about 0.1 to about 5,000 ppm; and in still other applications from about 100 to about 2,500 ppm. The more colorant present, the darker the shade therein.

The term “thermoset” or “thermosets” refers to a polymeric solid which upon exposure to sufficient heat or in the presence of a sufficient amount of catalyst, configures itself into a pre-determined shape. Thus, foams, sheets, articles, coverings, and the like, are all possible, and within the scope of the invention.

The inventive colorant compounds may be added in any amount to such thermosets up to their saturation limits. The amount may be between about 0.00001 ppm to about 25,000 ppm per total amount of resin; in other aspects, may be from about 0.001 to about 15,000 ppm; in other applications may be between about 0.1 to about 5,000 ppm. The more colorant present, the darker the shade therein. When mixed with other colorants within the target thermoset, the same amounts may be used within the saturation limit, i.e. dependent upon the amount of any extra colorants therein.

Thermoplastic and/or thermoset colorants (and other additives) are typically added to such compositions during the injection molding (or other type of molding, such as blow molding), including, and without limitation, by mixing the liquid absorber with resin pellets and melting the entire coated pellets, or through a masterbatch melting step while the resin and absorber are pre-mixed and incorporated together in pellet form. Such plastics include for example polyolefins, polyesters, polyamides, polyurethanes, polycarbonates, and other well known resins. Generally, such plastics, including the colorant, UV absorber, and other potential additives, are formed through any number of various extrusion techniques. Thermoplastics may include polyesters, such as PET (polyethylene terephthalate). “Plastic packaging” encompasses containers, sheets, blister packages, and the like, utilized for storage purposes and which include the plastics in any combination as noted above.

The term “pure, undiluted state” as used in conjunction with the inventive colorant compounds indicates that the compounds themselves without any additives are liquid at room temperature. Thus, there may be no need to add solvents, viscosity modifiers, and other like additives to the compounds to effectuate such a desirable physical state.

The colorant compounds may be liquid in nature at ambient temperature and pressure and at substantial purity; however—pasty, waxy, or crystalline colorants also are contemplated within this invention. To effectuate coloring of substrates and media, any other standard colorant additives, such as resins, preservatives, surfactants, solvents, antistatic compounds, antioxidants, antimicrobials may also be utilized within the inventive colorant compound compositions or methods.

For liquid composition applications, the amount present may range from about 0.00001 ppm to about 30,000 ppm of the total solvent present; or from about 0.001 to about 15,000 ppm; or in other applications from about 0.1 to about 5,000 ppm; and also about 100 to about 2,500 ppm.

### Lactone-Derived, Lactam-Derived, and thiolactone-Derived Compounds

There are numerous compounds that may be employed in the practice of the invention, not limited to those set forth below. Below are several classes of compounds that may have application in the practice of the invention.

In examples 1, 3, 4, 6, 7, 9, 10, 12, 13, and 15, R₁-R₈ may be the same or different and selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives.

In examples 5, 8 11 nd 14, R₁ and R₂ may be the same or different and may be selected from hydrogen, alkyl, aryl, acetoxy, benzyl, acyl, silyl, cycloalkyl, allyl, alkenyl, polyoxyalkylene, oxyalkylene, and alkynyl; and R₃, R₄, R₅, R₆, R₇, and R₈ may be the same or different and may be selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives. Tautomeric forms of the lactam ring (in which the carbonyl group exists in the enol form) are also envisioned as part of this invention.

### Example 1

### Benzene-Centered Dilactones

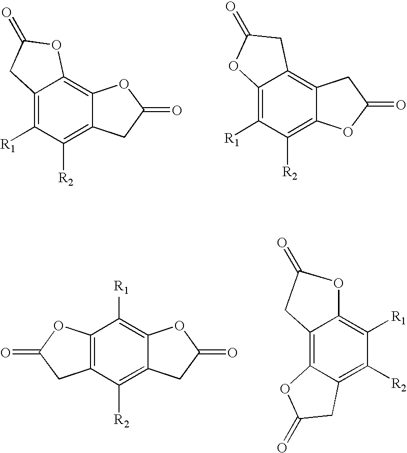

### Example 2A

### Benzene-Centered Dilactams

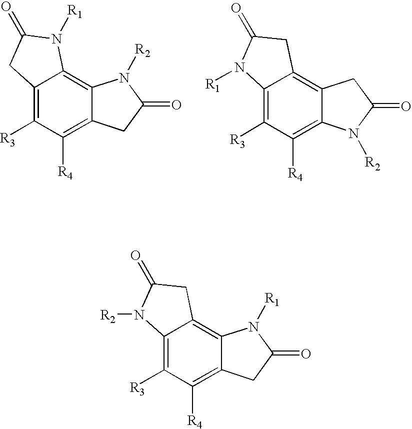

The above compounds may include groups as defined herein. R₁ and R₂ may be the same or different and may be selected from hydrogen, alkyl, aryl, acetoxy, benzyl, acyl, silyl, cycloalkyl, allyl, alkenyl, polyoxyalkylene, oxyalkylene, and alkynyl; R₃ and R₄ may be the same or different and may be selected from C₁₋₂₀ alkyl, alkylester, halogen, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives. Tautomeric forms of the lactam ring (in which the carbonyl group exists in the enol form) are also envisioned as part of this invention.

### Example 2B

### Benzene-Centered Dilactams

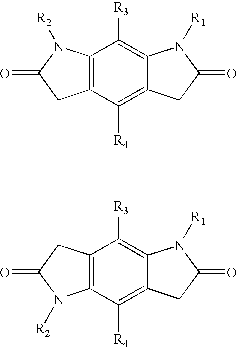

The above compounds may include groups as defined herein. R₁ and R₂ may be the same or different and may be selected from hydrogen, alkyl, aryl, acetoxy, benzyl, acyl, silyl, cycloalkyl, allyl, alkenyl, polyoxyalkylene, oxyalkylene, and alkynyl R₃ and R₄ may be the same or different and may be selected from C₁₋₂₀ alkyl, alkylester, halogen, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives; wherein if both R₁ and R₂ are both hydrogen then R₃ and R₄ may be the same or different and selected from C₁₋₂₀ alkyl, alkylester, halogen, hydroxyl, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives; and if R₃ and R₄ are both hydrogen then R₁ and R₂ may be the same or different and may be selected from alkyl, aryl, acetoxy, benzyl, acyl, silyl, cycloalkyl, allyl, alkenyl, polyoxyalkylene, oxyalkylene, and alkynyl. Tautomeric forms of the lactam ring (in which the carbonyl group exists in the enol form) are also envisioned as part of this invention.

### Example 3

### Benzene-Centered Dithiolactone

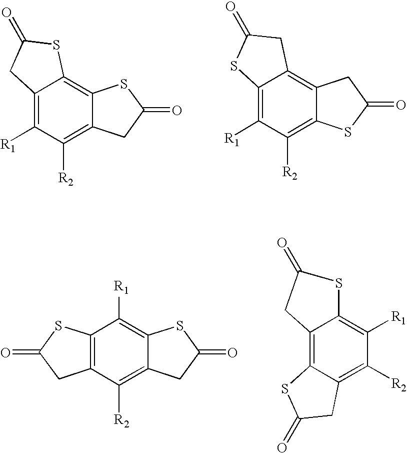

### Example 4

### Naphthalene-Centered Dilactones

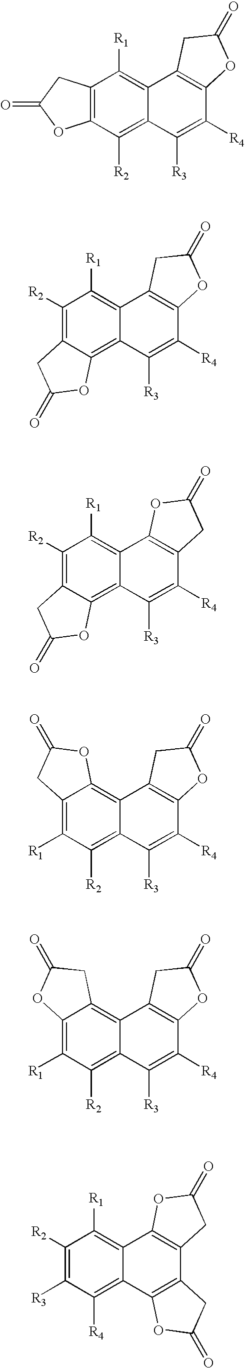

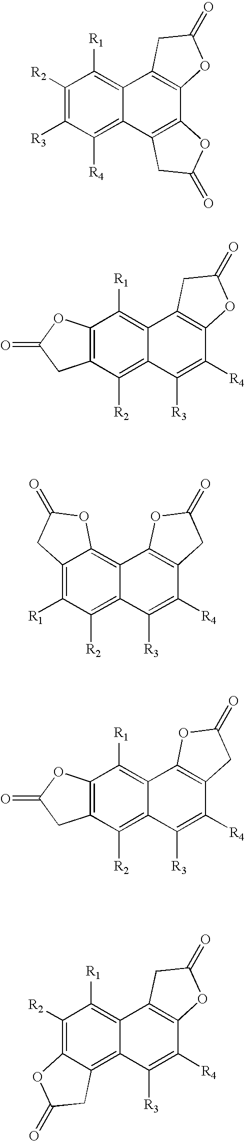

### Example 5

### Naphthalene-Centered Dilactams

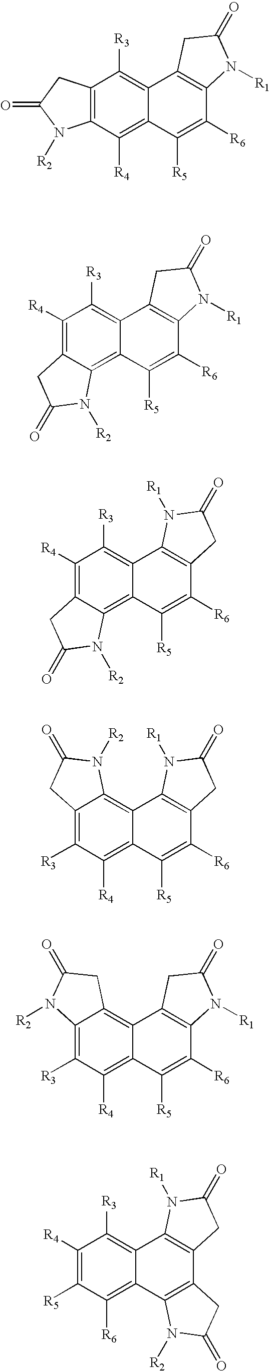

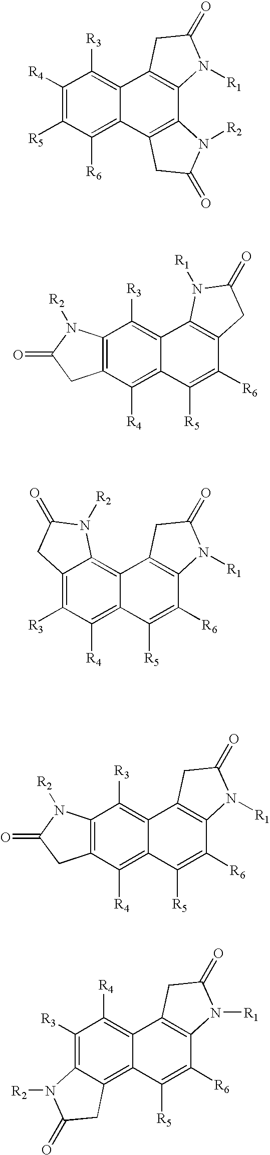

### Example 6

### Naphthalene-Centered Dithiolactones

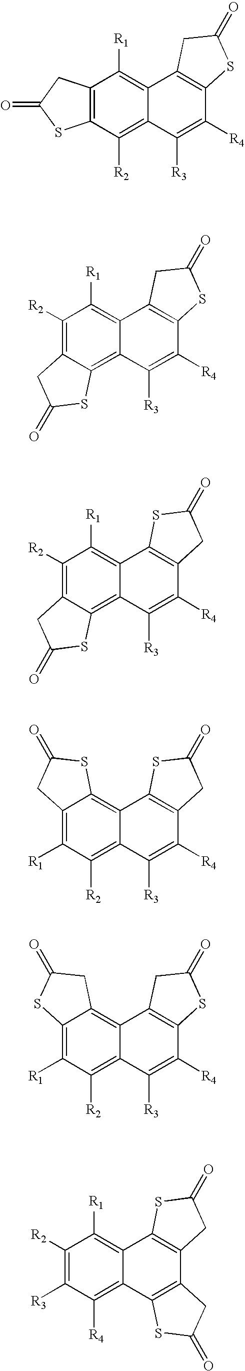

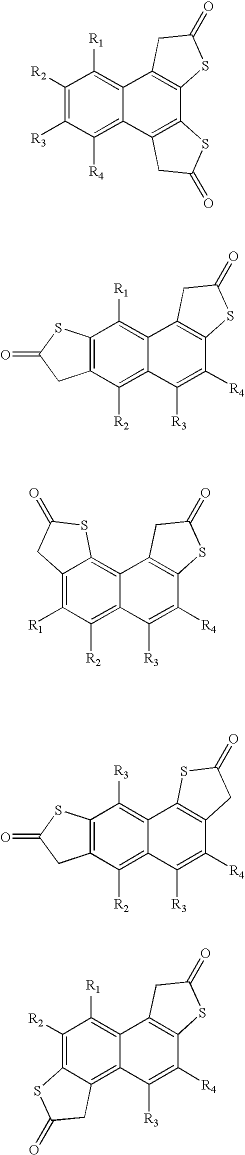

### Example 7

### Anthraquinone-Centered Dilactones

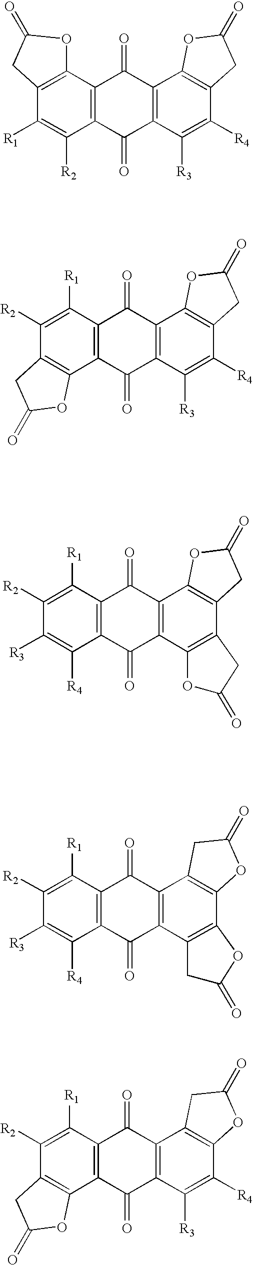

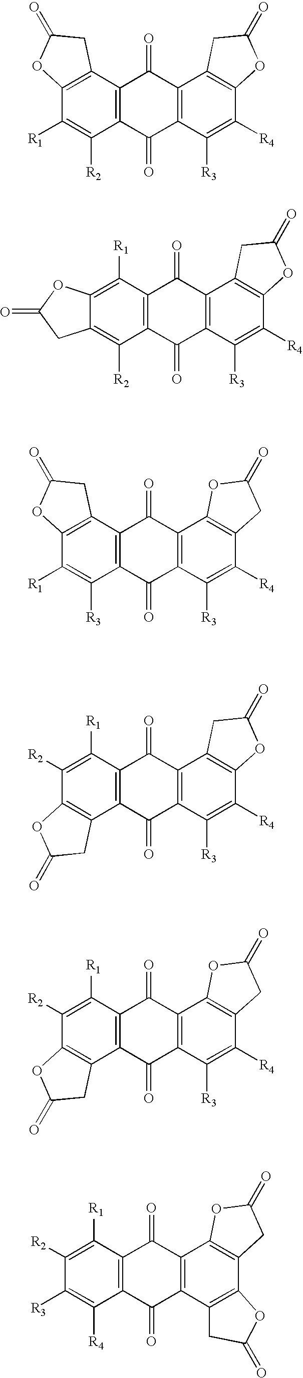

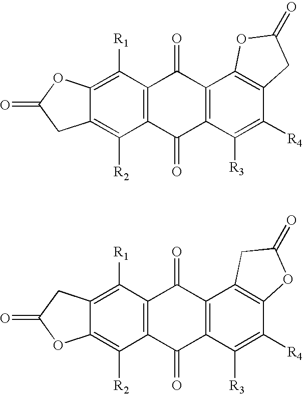

### Example 8

### Anthraquinone-Centered Dilactams

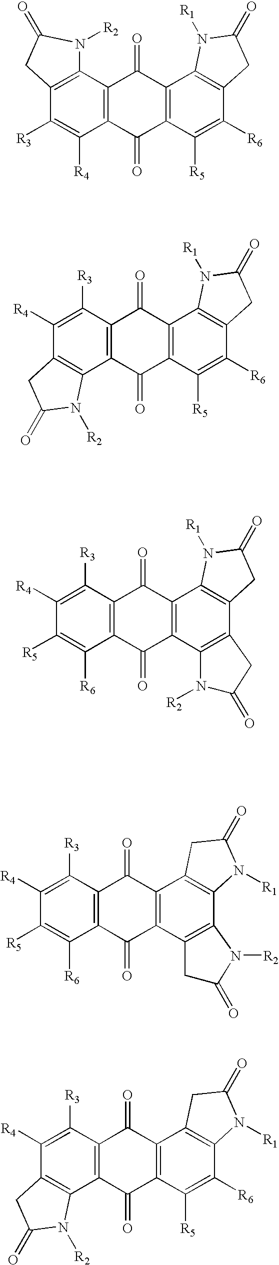

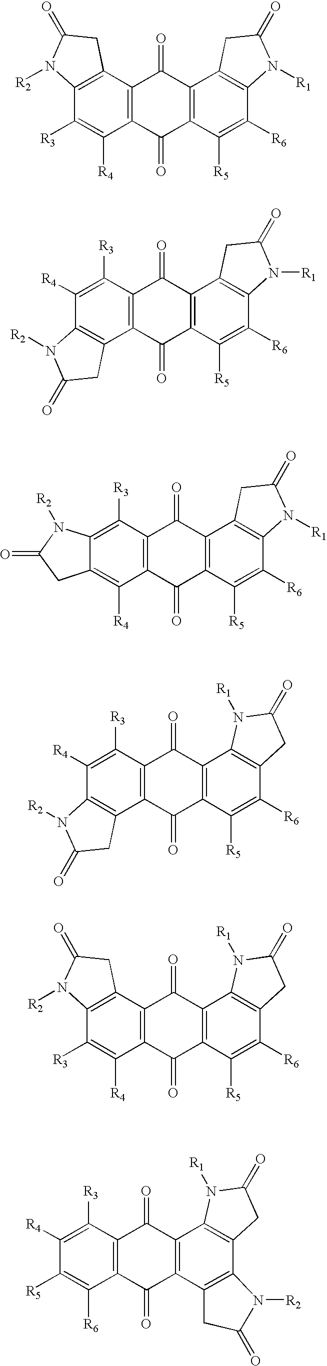

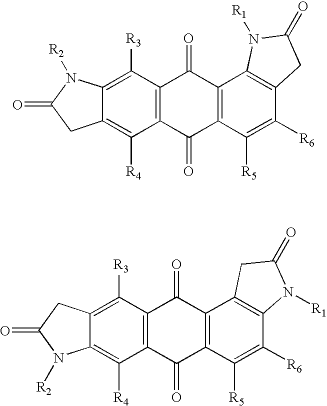

### Example 9

### Anthraquinone-Centered Dithiolactones

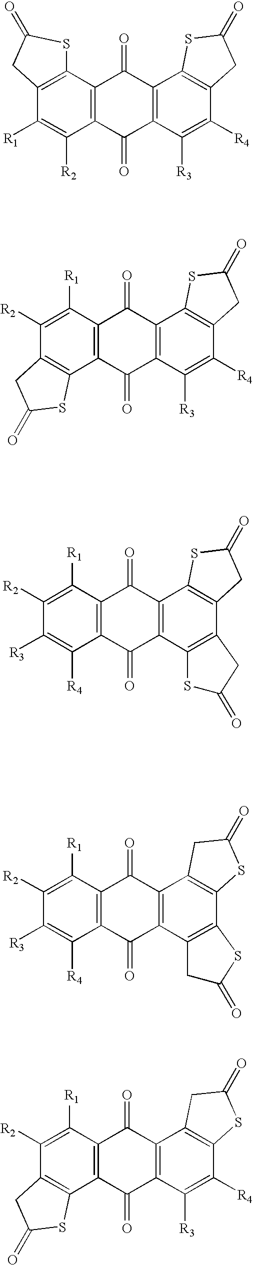

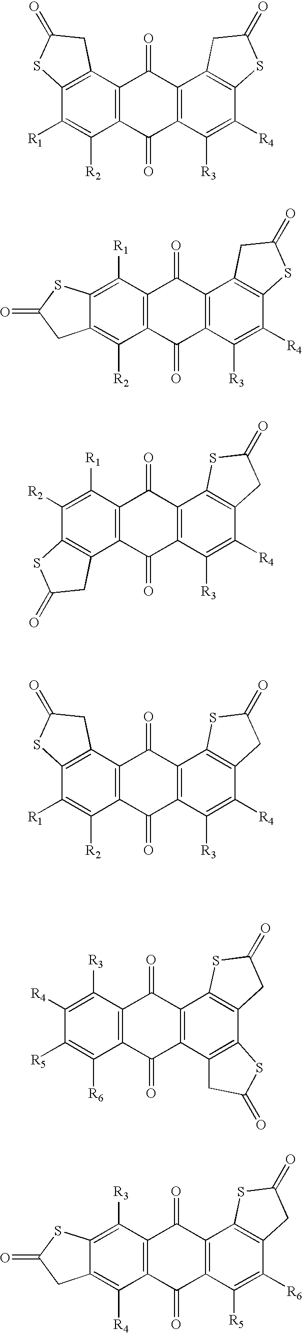

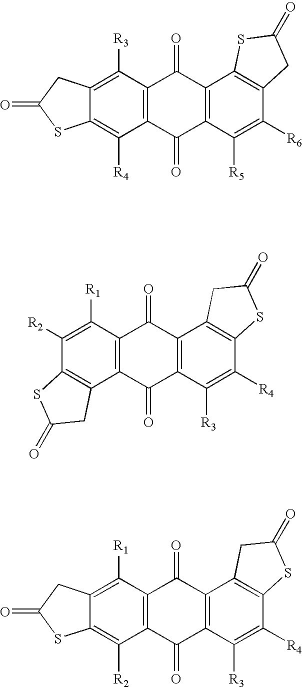

### Example 10

### Anthracene-Centered Dilactones

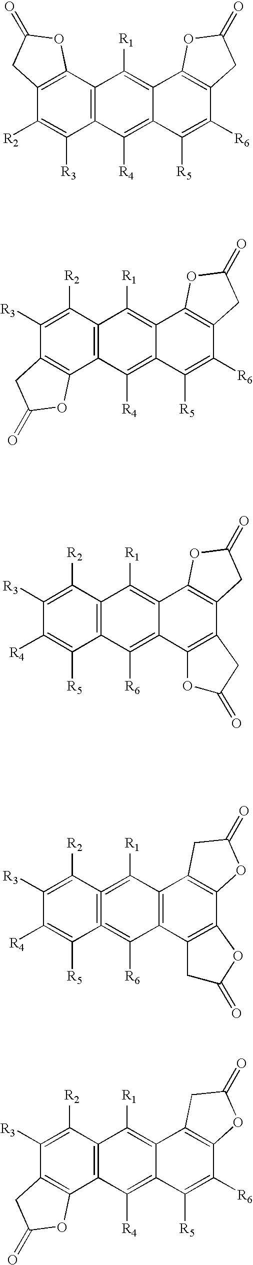

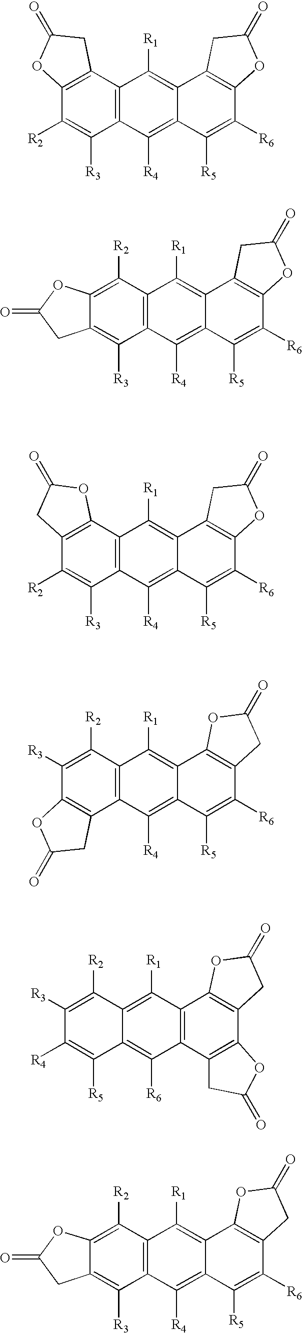

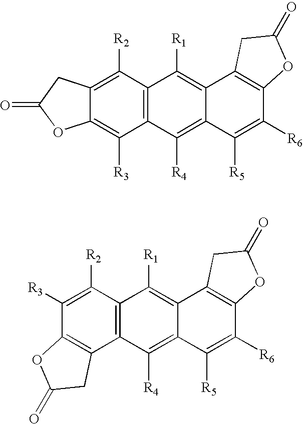

### Example 11

### Anthracene-Centered Dilactams

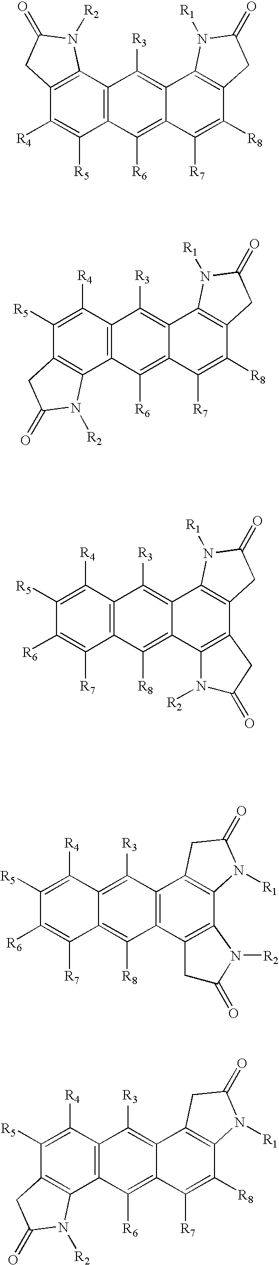

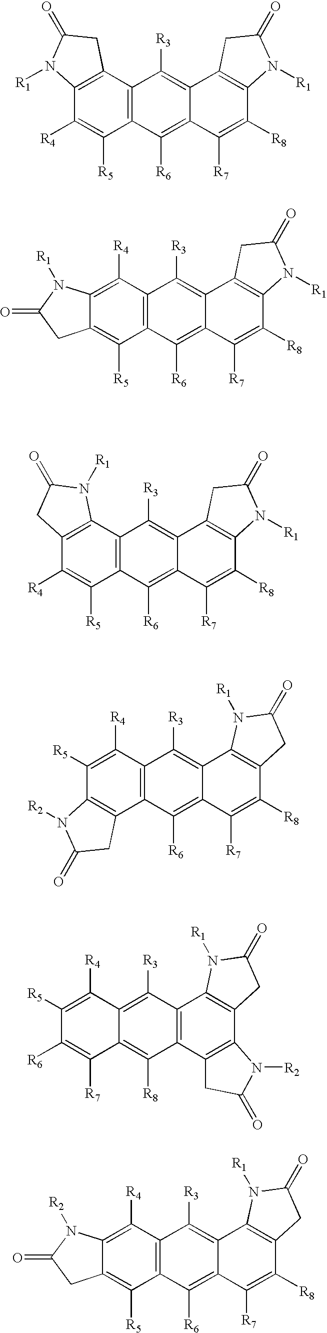

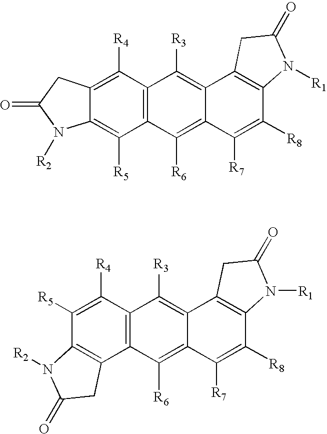

### Example 12

### Anthracene-Centered Dithiolactones

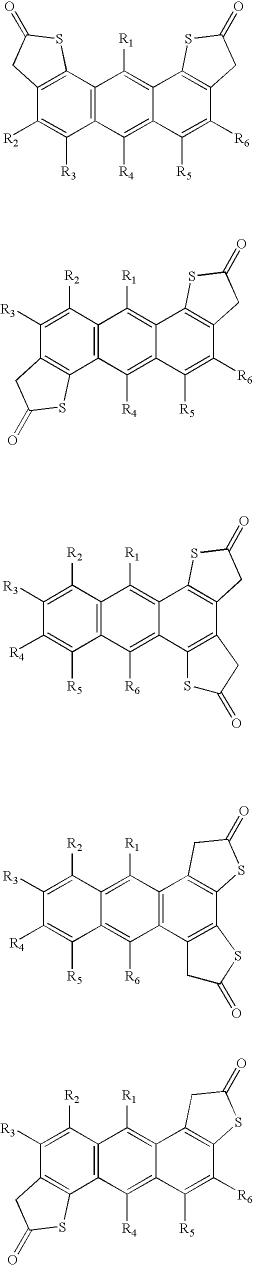

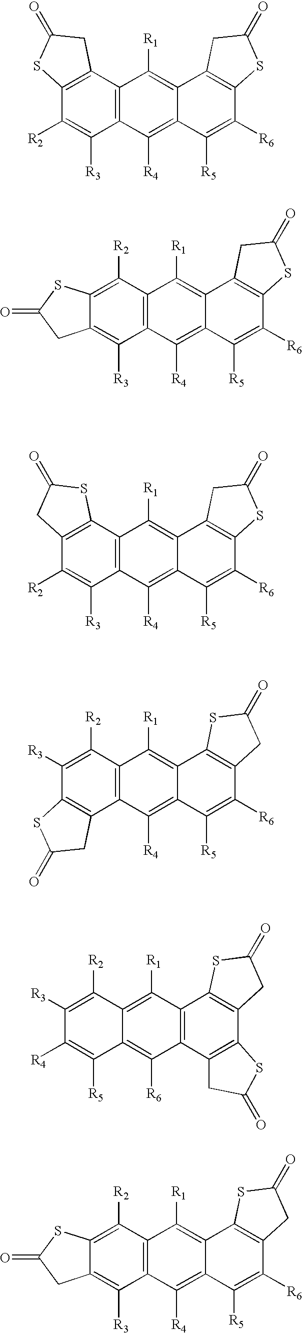

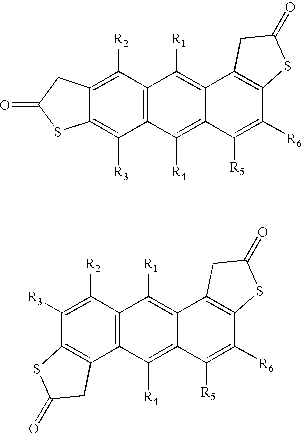

### Example 13

### Hetero-Aromatic-Centered Dilactones

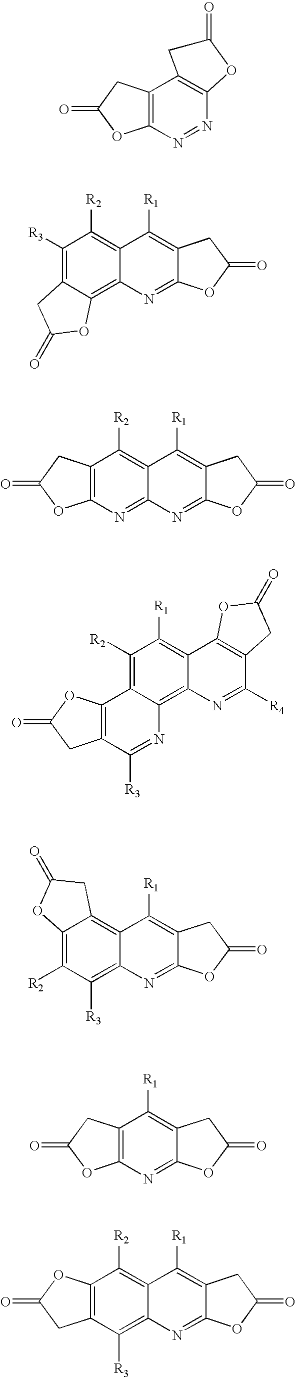

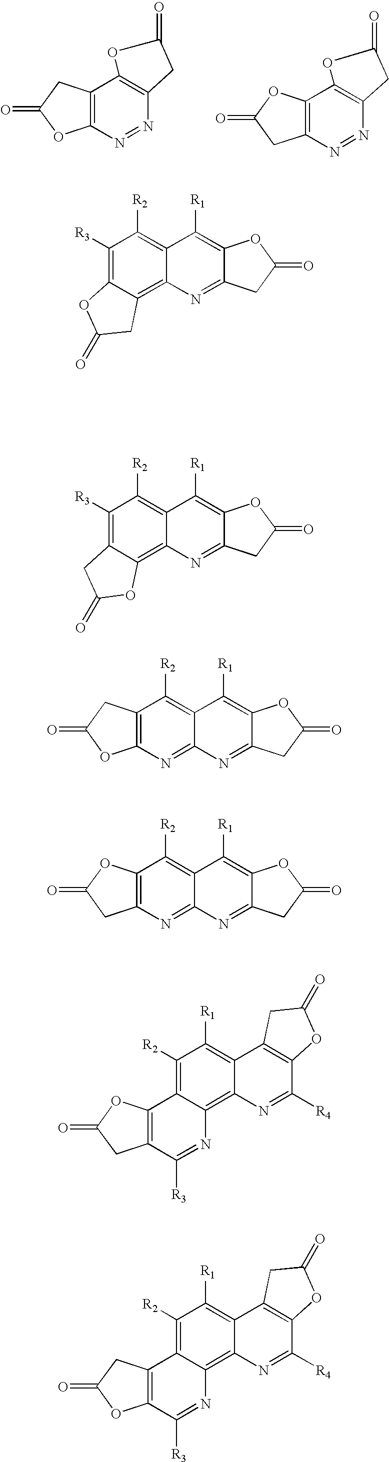

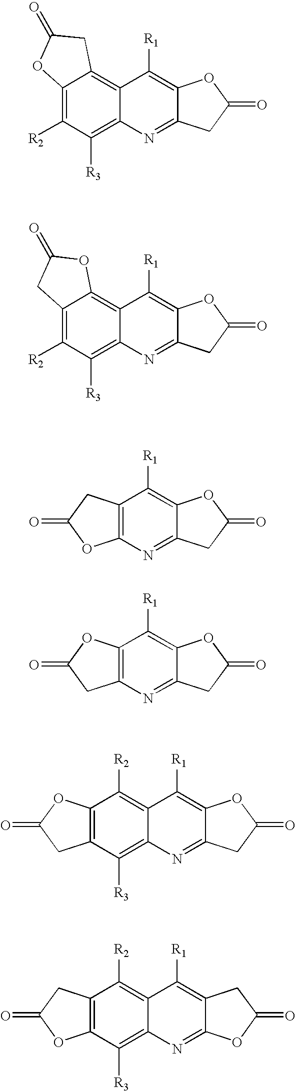

### Example 14

### Hetero-Aromatic-Centered Dilactams

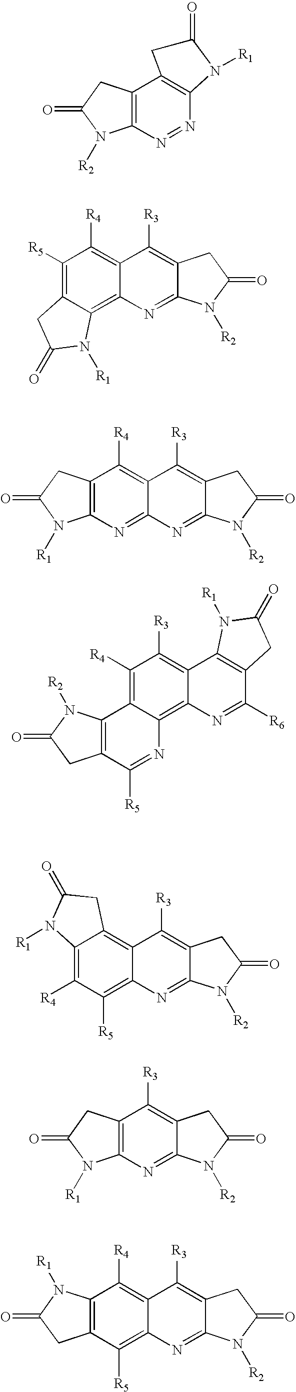

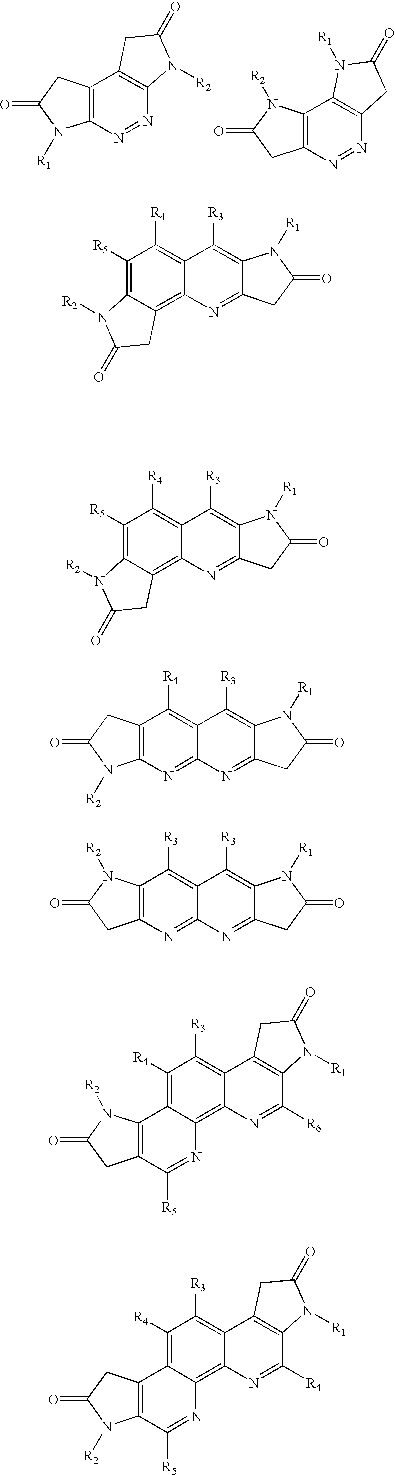

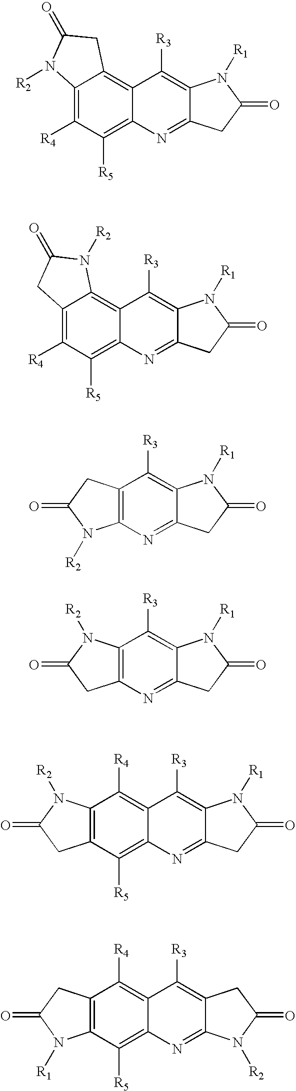

### Example 15

### Hetero-Aromatic-Centered Dithiolactones

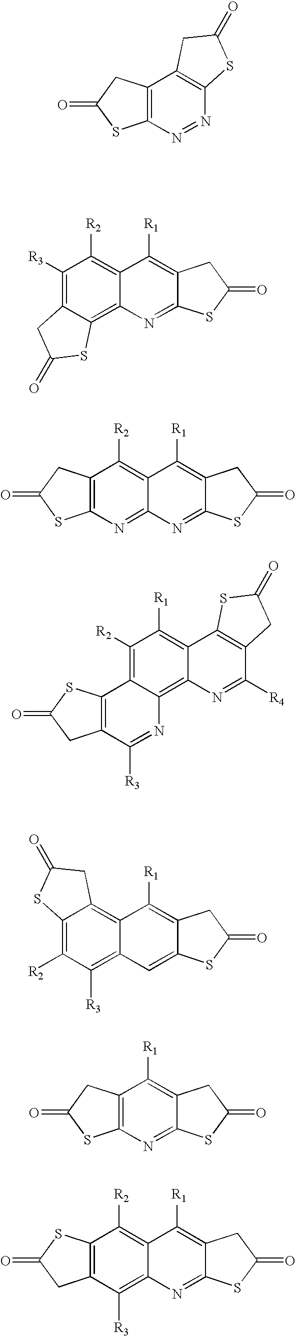

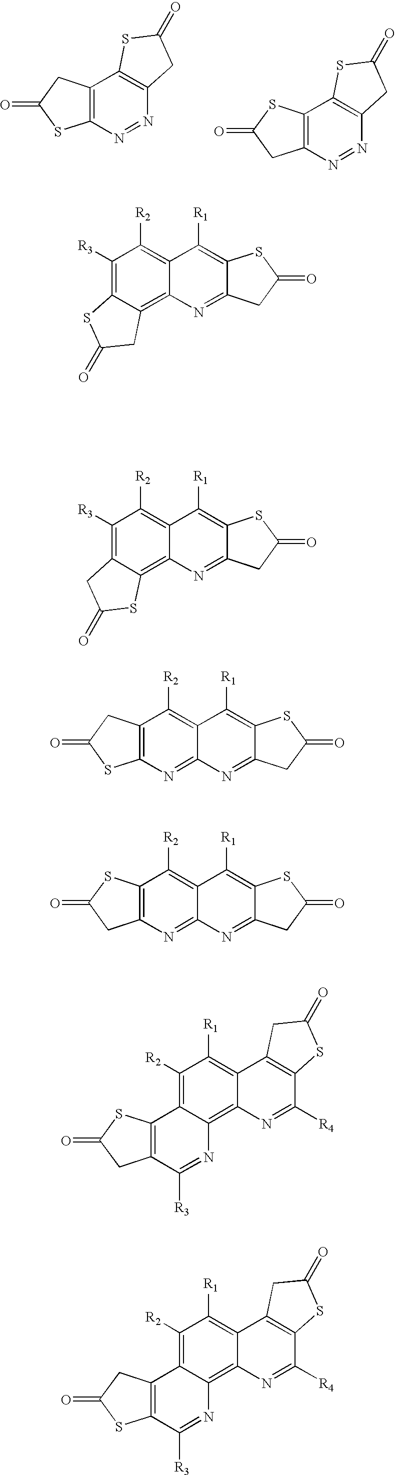

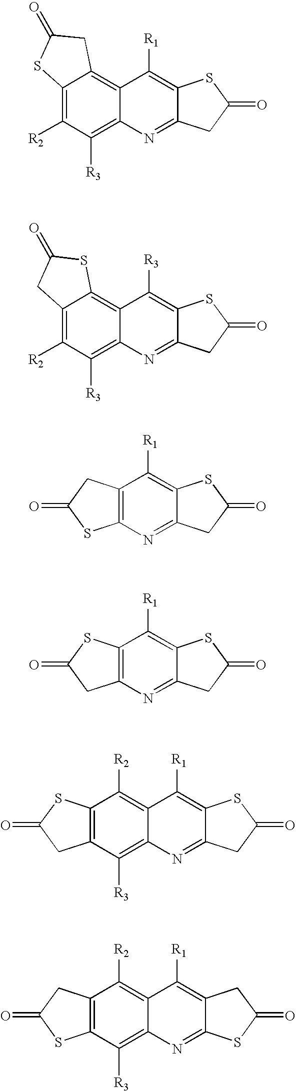

### Lactone-Derived, Lactam-Derived, and Thiolactone-Derived Bismethine Compounds (BLA Bismethines)

There are numerous compounds that may be employed in the practice of the invention, not limited to those set forth below. Below are several classes of compounds that may have application in the practice of the invention.

In examples 16, 19, 20, 22, 23, 25, 26, 28, 29, and 31 R₁-R₈ can be the same or different and selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives; and A and B may be the same or different and selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives.

The geometry of the methine double bond in examples 16-31 is not intended show restriction to cis or trans geometry.

In examples 17, 21, 24, 27, and 30 R₁ and R₂ may be the same or different and may be selected from hydrogen, alkyl, aryl, acetoxy, benzyl, acyl, silyl, cycloalkyl, allyl, alkenyl, polyoxyalkylene, oxyalkylene, and alkynyl; R₃, R₄, R₅, R₆, R₇, and R₈ may be the same or different and may be selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, substituted naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives. Tautomeric forms of the lactam ring (in which the carbonyl group exists in the enol form) are also envisioned as part of this invention. A and B may be the same or different and selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, substituted naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives.

### Example 16

### Benzene-Centered Dilactones

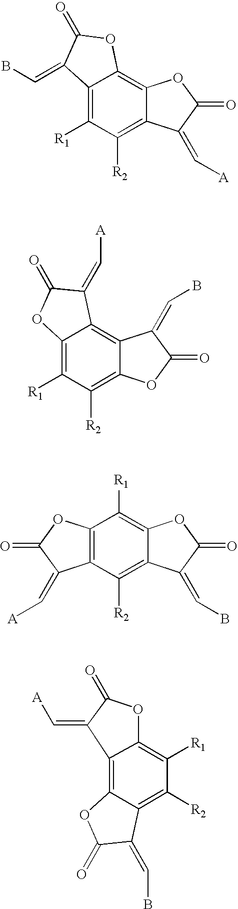

### Example 17

### Benzene-Centered Dilactam Bismethines

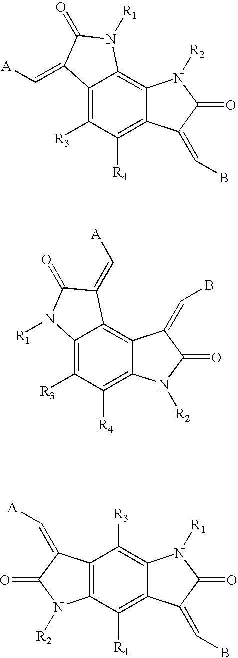

### Example 18

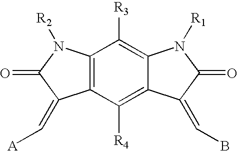

The above compound may include groups as defined herein. R₁ and R₂ may be the same or different and may be selected from hydrogen, alkyl, aryl, acetoxy, benzyl, acyl, silyl, cycloalkyl, allyl, alkenyl, polyoxyalkylene, oxyalkylene, and alkynyl; R₃ and R₄ may be the same or different and may be selected from C₁₋₂₀ alkyl, alkylester, halogen, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, substituted naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives. Tautomeric forms of the lactam ring (in which the carbonyl group exists in the enol form) are also envisioned as part of this invention, and A and B may be the same or different and selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, aryl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, substituted naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives wherein if R₁, R₂, R₃, R₄ are all hydrogen then A and B is selected from C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, sulphonylamino, acyl, substituted aryl, heteroaryl, allyl, alkenyl, alkynyl, oxyalkylene, polyoxyalkylene, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, substituted naphthyl, polyoxyalkyleneamino substituted aryl, polyoxyalkylene substituted aryl, aniline derivatives, 2,5-dimethoxyaniline derivatives, phenol derivatives, polyoxyalkylene substituted aniline derivatives, and polyoxyalkylene substituted phenol derivatives.

### Example 19

### Benzene-Centered Dithiolactone Bismethines

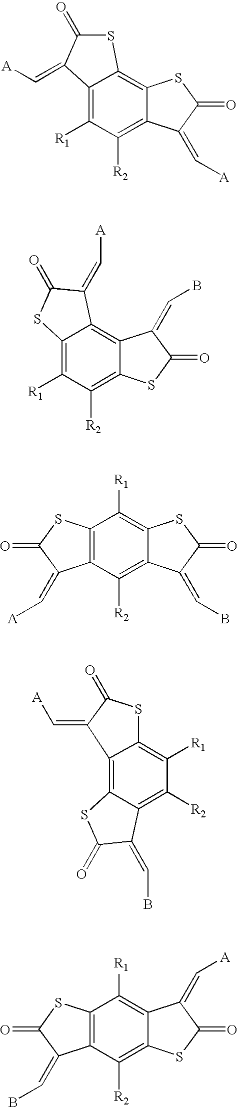

### Example 20

### Naphthalene-Centered Dilactone Bismethines

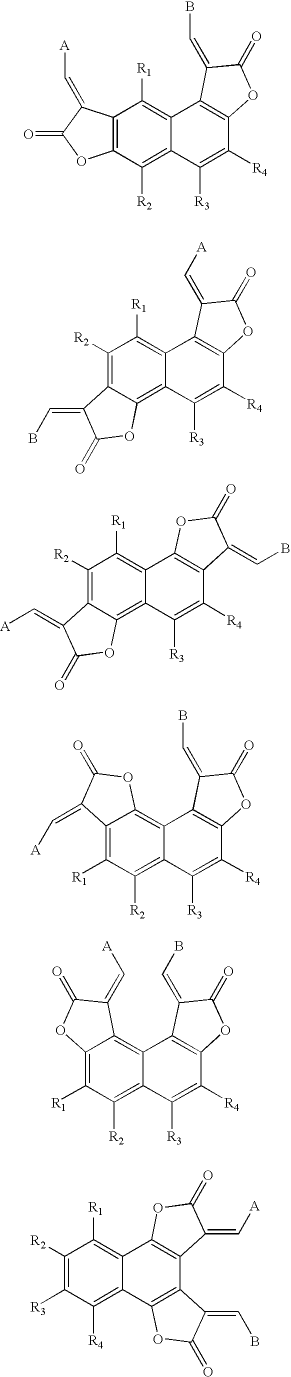

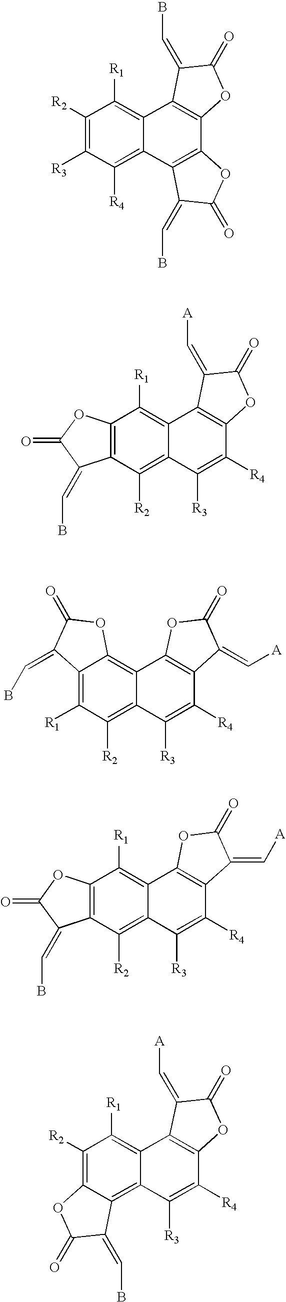

### Example 21

### Naphthalene-Centered Dilactams

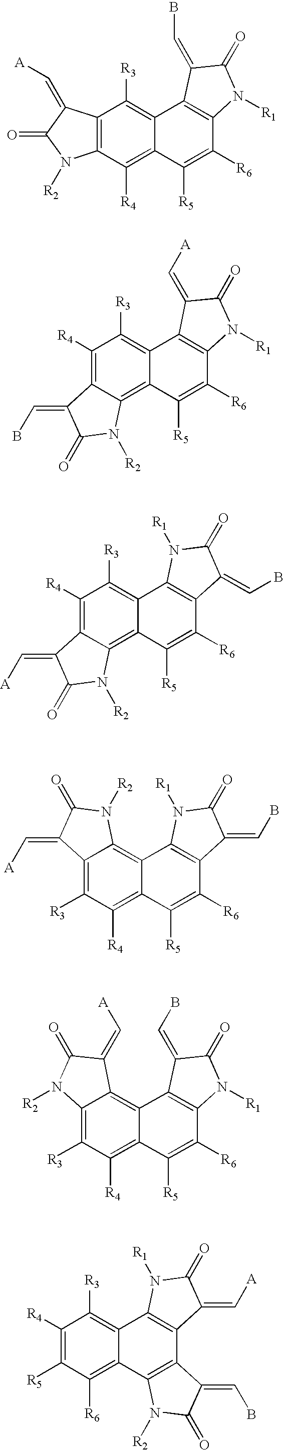

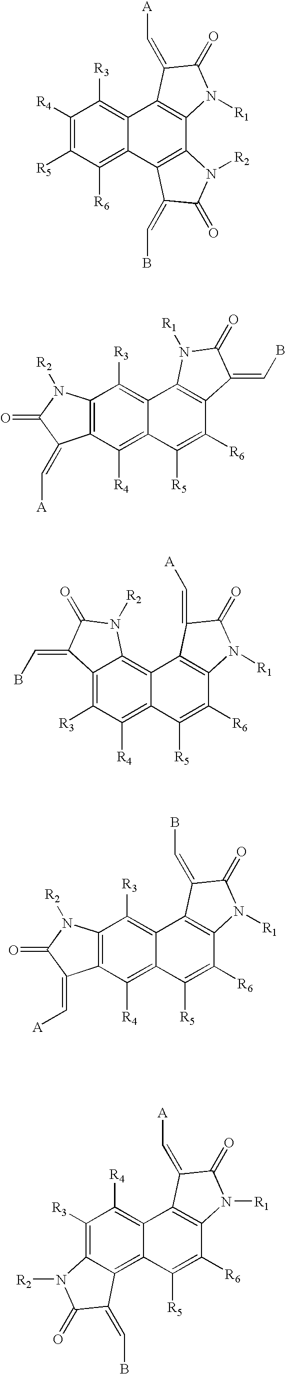

### Example 22

### Naphthalene-Centered Dithiolactones

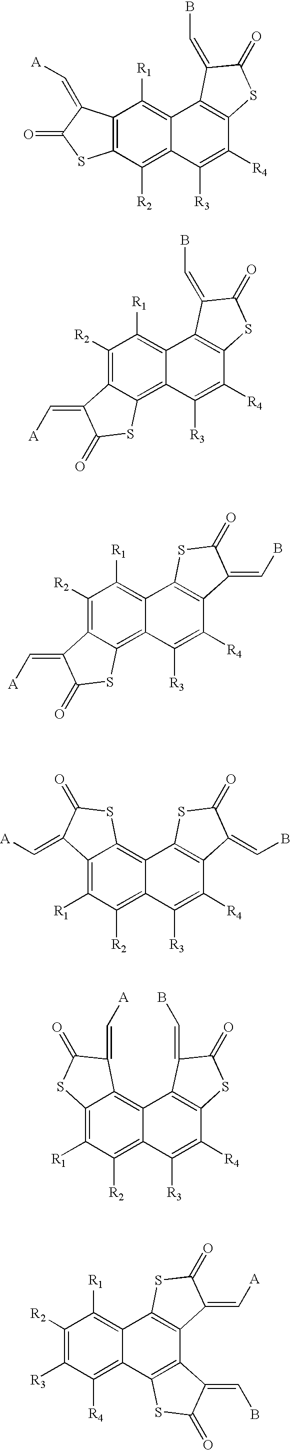

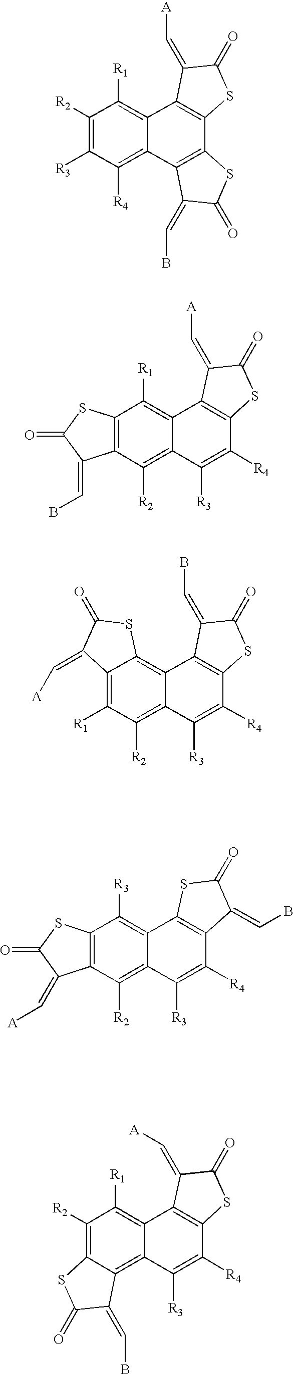

### Example 23

### Anthraquinone-Centered Dilactones

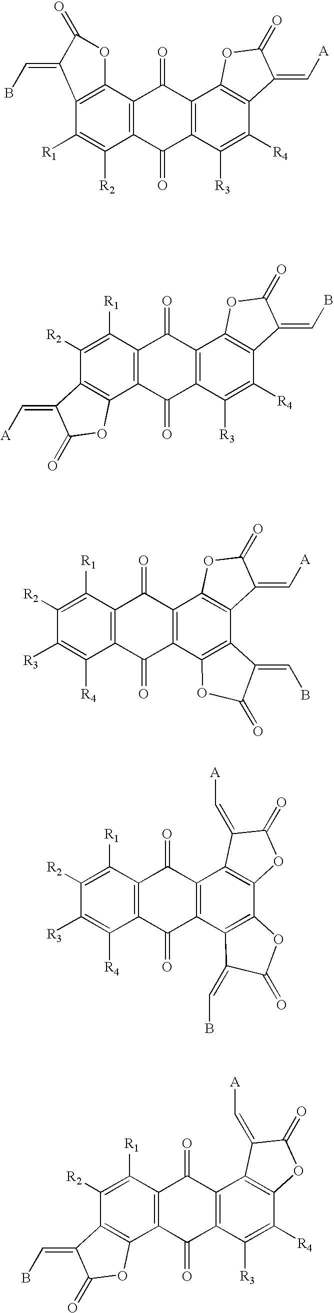

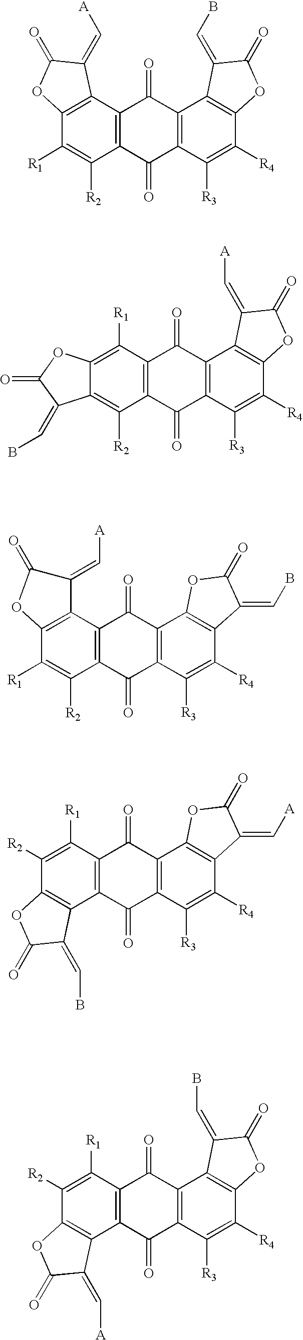

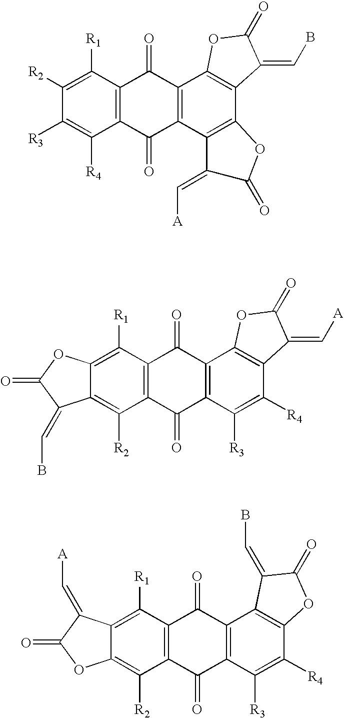

### Example 24

### Anthraquinone-Centered Dilactams

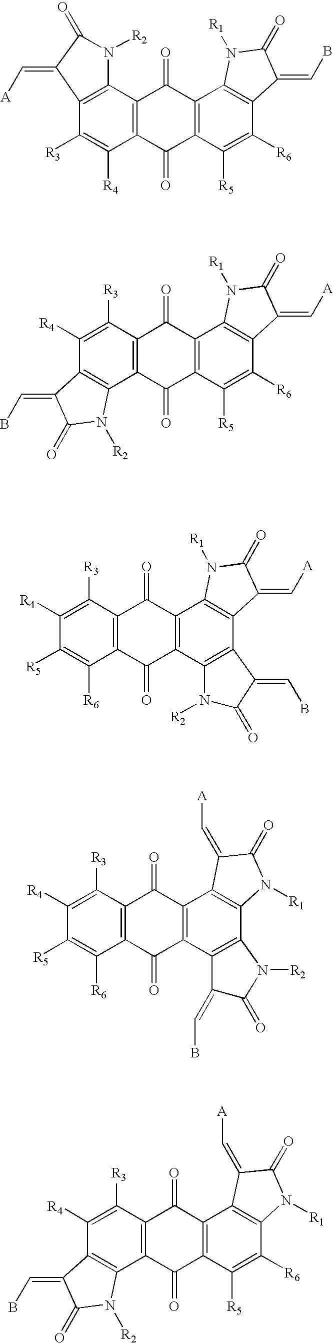

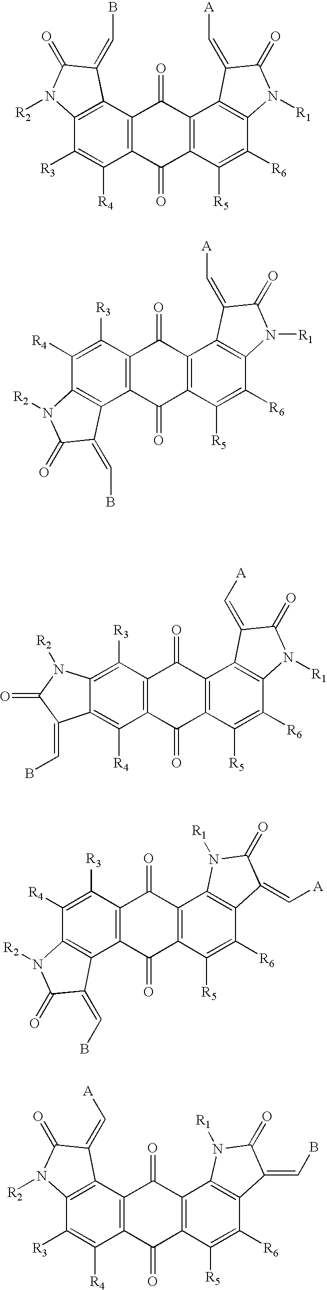

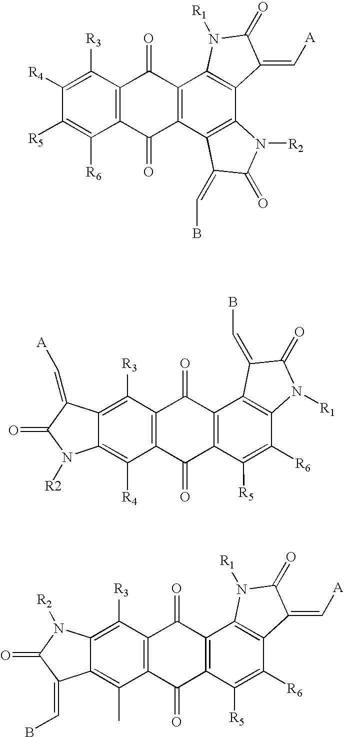

### Example 25

### Anthraquinone-Centered Dithiolactones

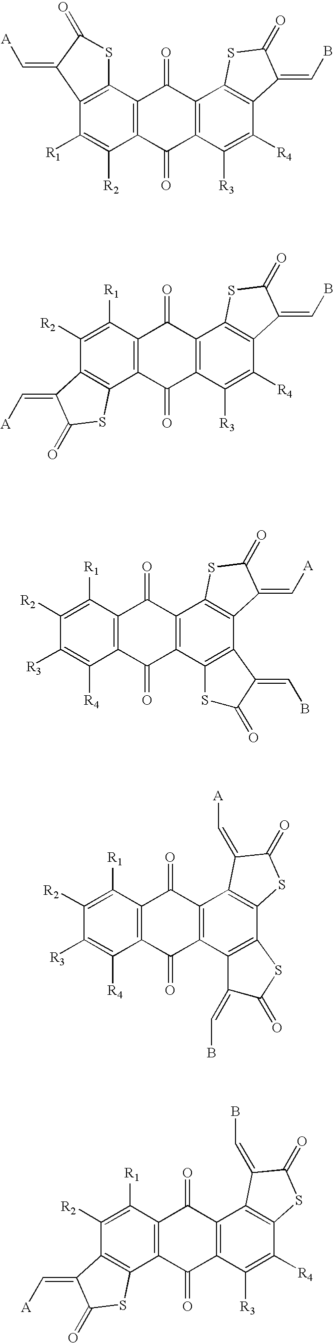

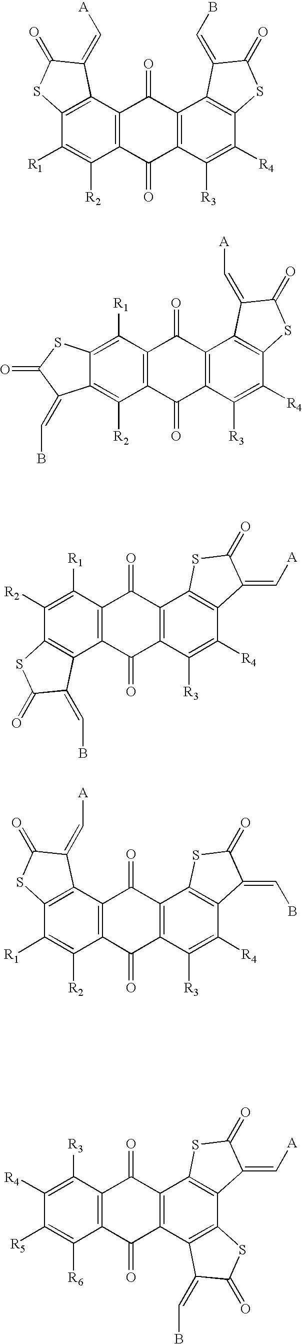

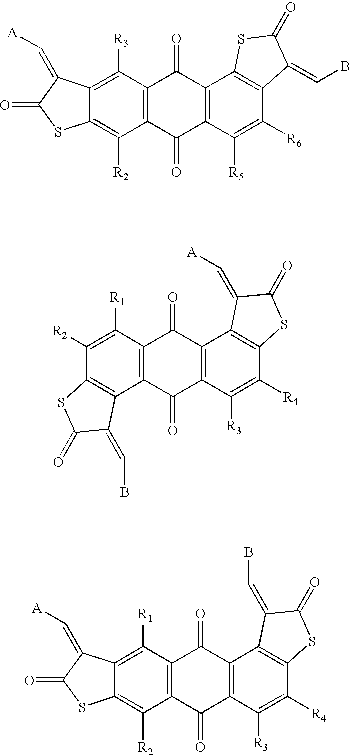

### Example 26

### Anthracene-Centered Dilactones

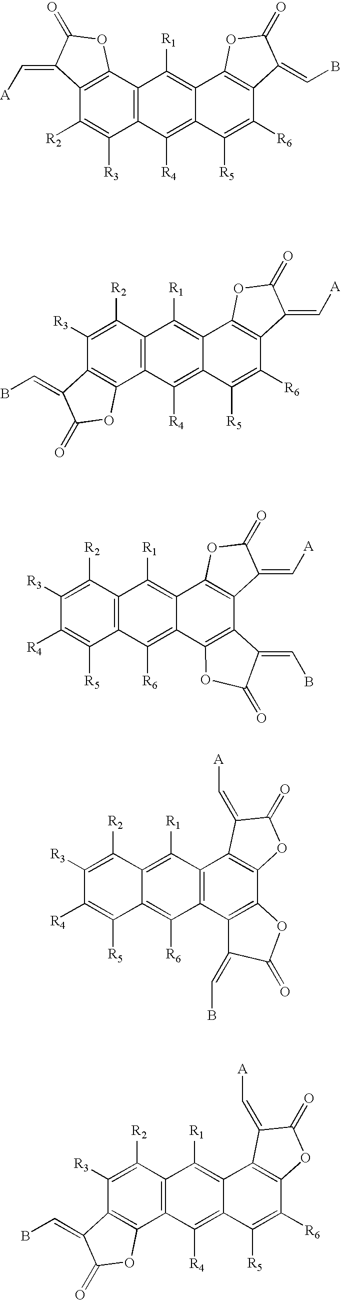

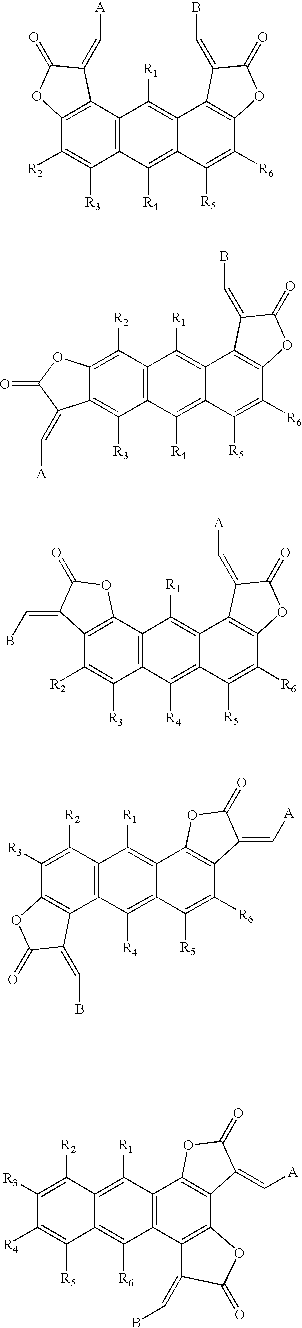

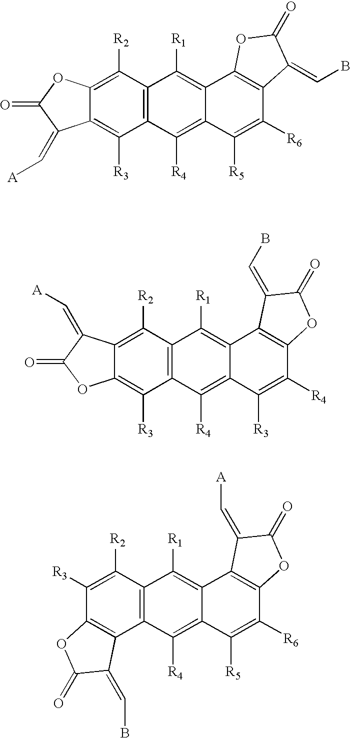

### Example 27

### Anthracene-Centered Dilactams

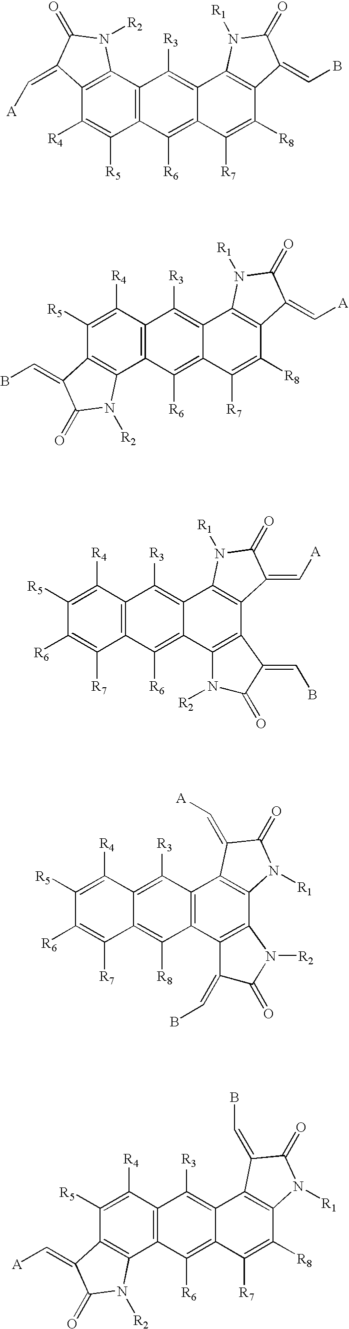

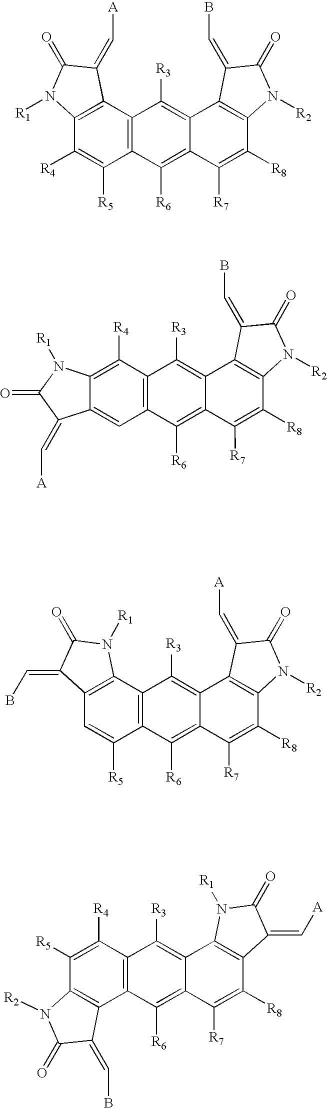

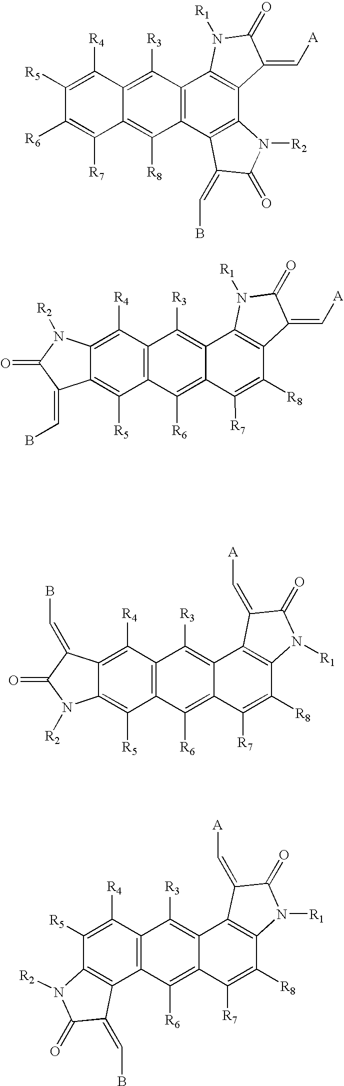

### Example 28

### Anthracene-Centered Dithiolactones

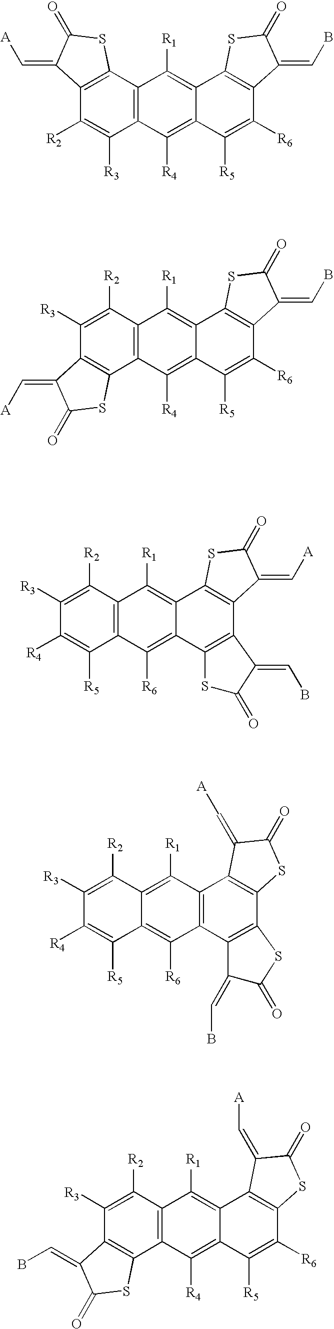

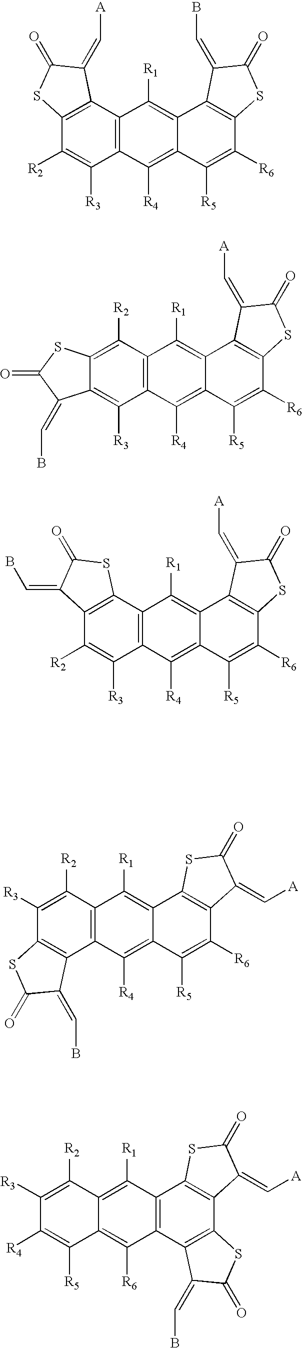

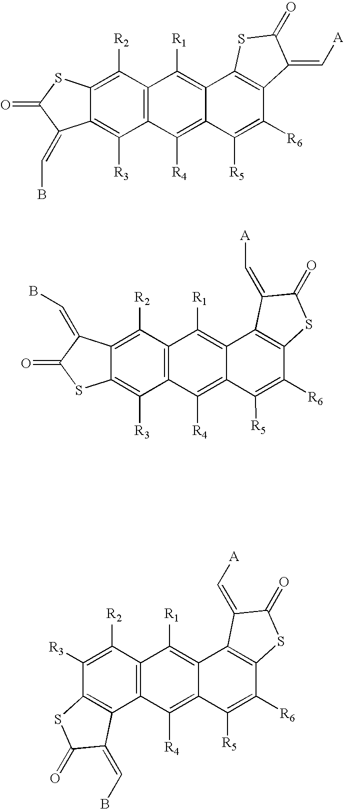

### Example 29

### Hetero-Aromatic-Centered Dilactones

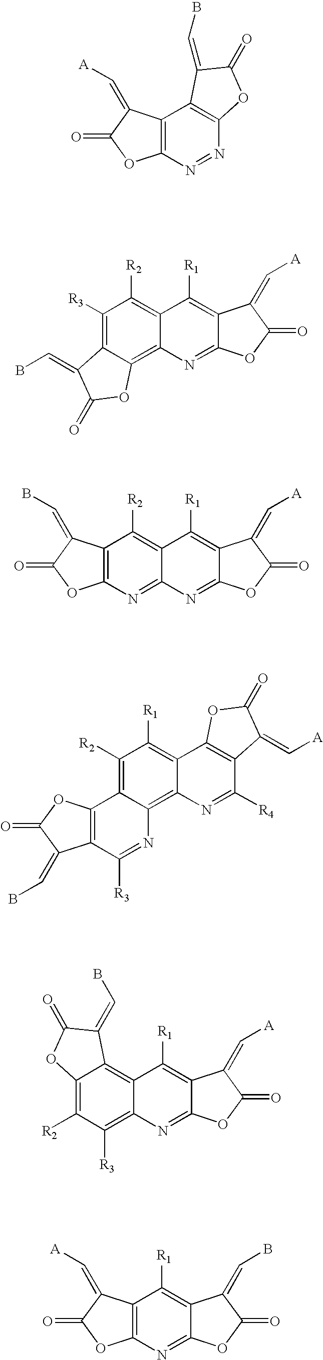

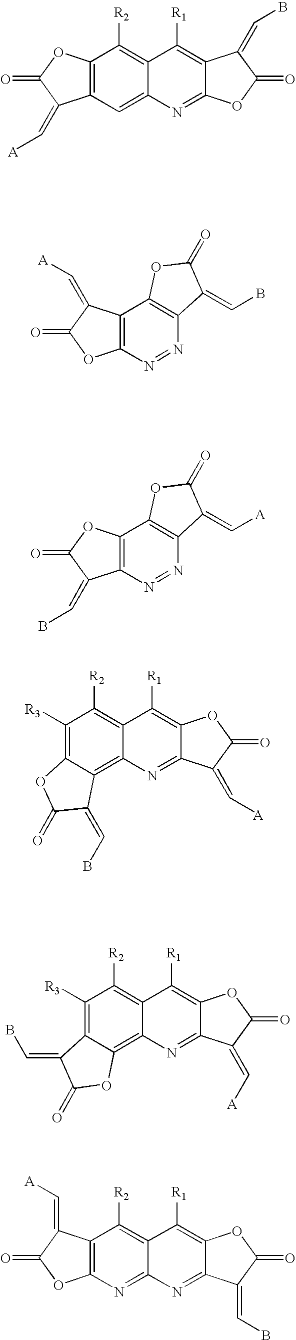

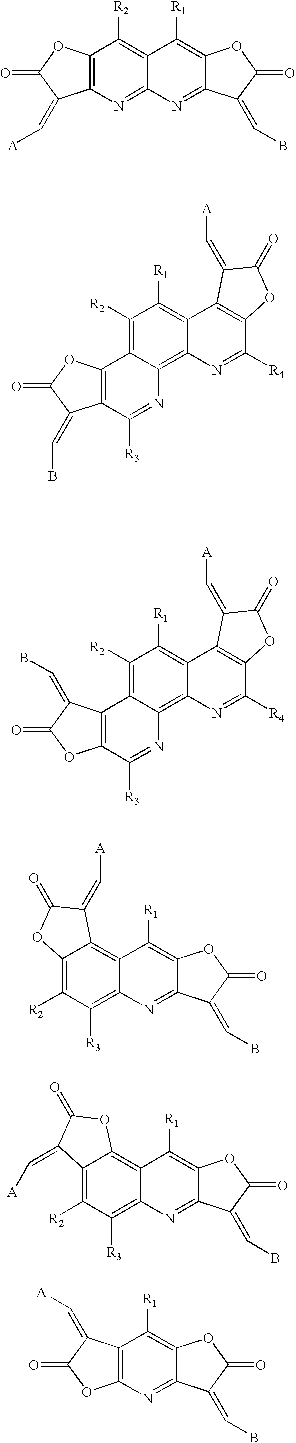

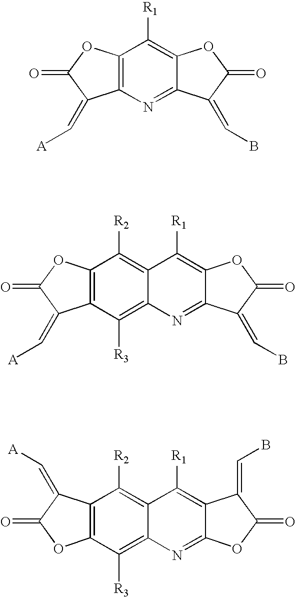

### Example 30

### Hetero-Aromatic-Centered Dilactams

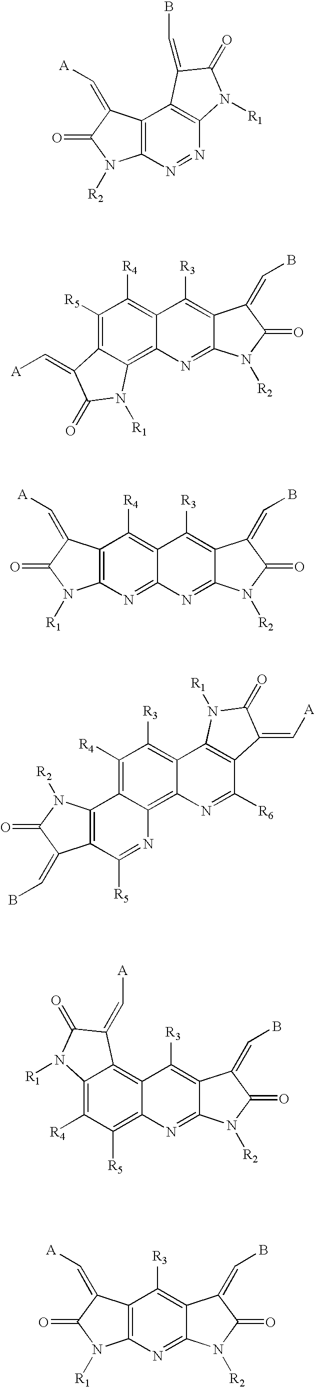

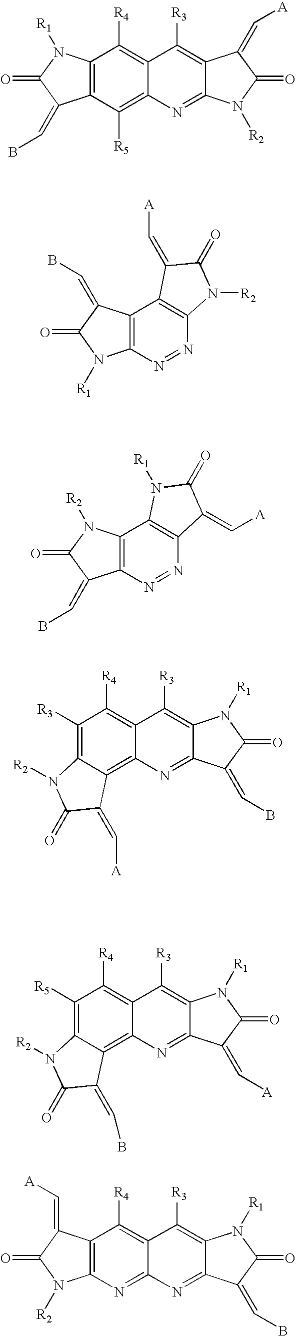

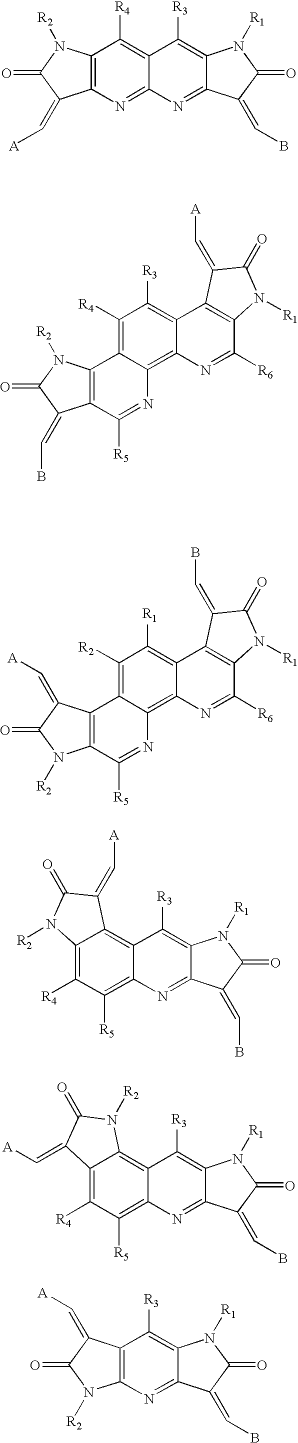

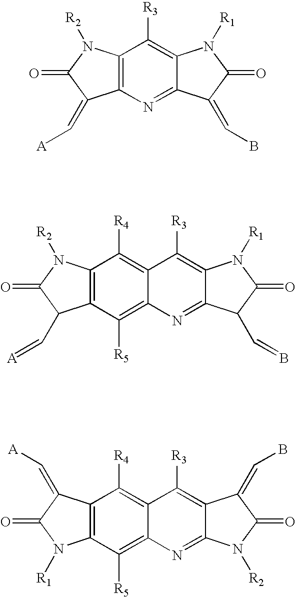

### Example 31

### Hetero-Aromatic-Centered Dithiolactones

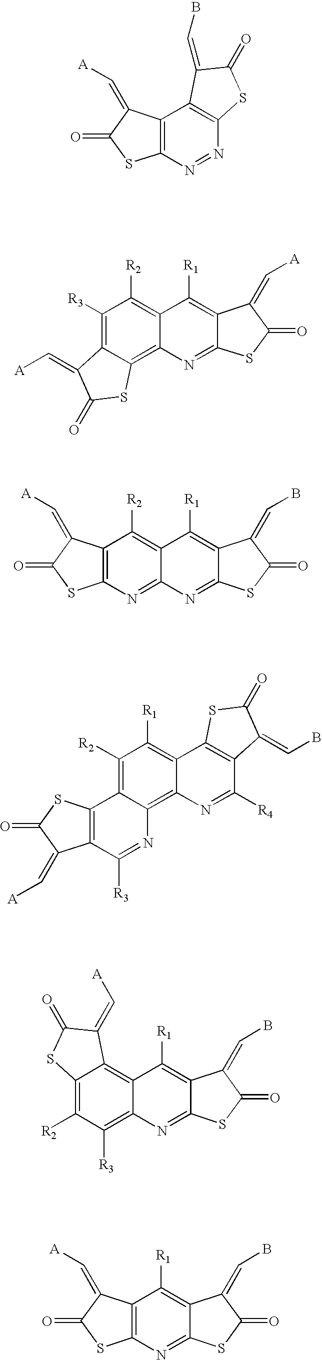

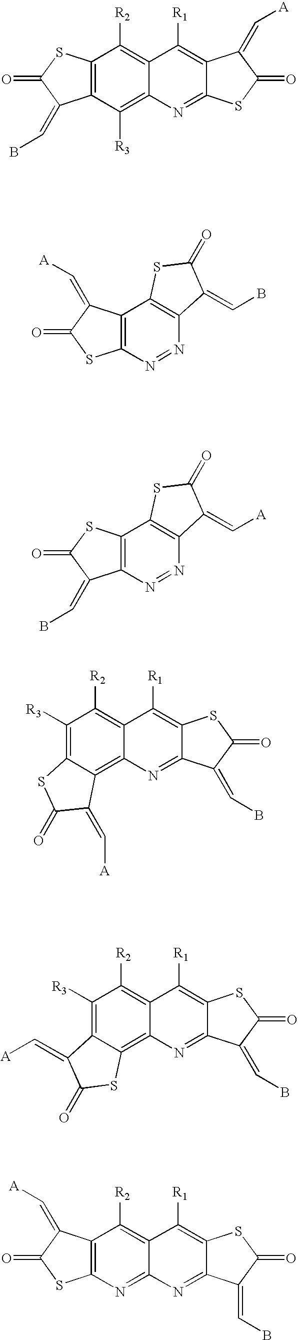

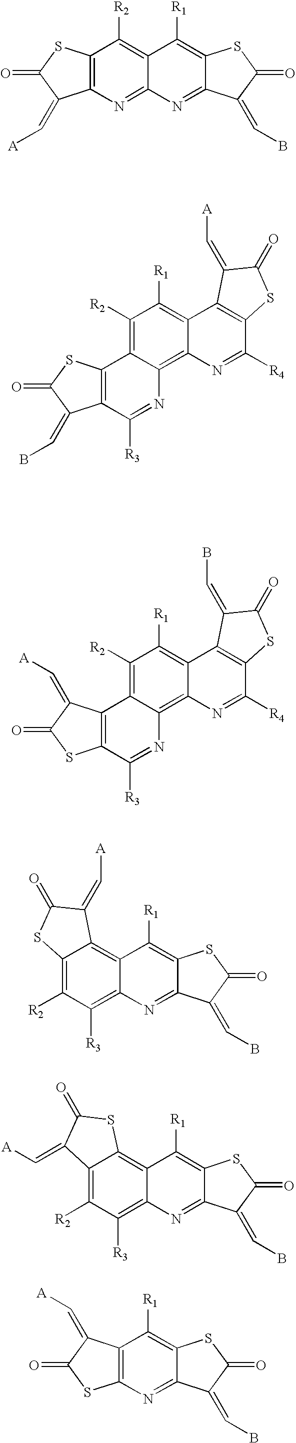

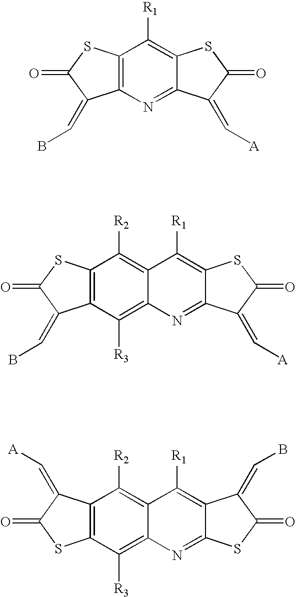

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims, and other compound species and genus may be contemplated within the scope of the invention as disclosed herein.

1. A benzene-centered lactone compound.

2. A benzene-centered lactam compound.

3. A benzene-centered thiolactone compound.

4. A naphthalene-centered lactone compound.

5. A naphthalene-centered lactam compound.

6. A naphthalene-centered thiolactone compound.

7. An anthraquinone-centered lactone compound.

8. An anthraquinone-centered lactam compound.

9. An anthraquinone-centered thiolactone compound.

10. An anthracene-centered lactone compound.

11. An anthracene-centered lactam compound.

12. An anthracene-centered thiolactone compound.

13. A hetero-aromatic-centered lactone compound.

14. A hetero-aromatic centered lactam compound.

15. A hetero-aromatic centered thiolactone compound.

16. A compound of the structure: 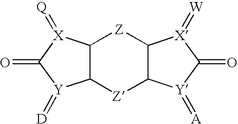
(a) wherein X, X′, Y, and Y′ are independently selected from the group consisting of: nitrogen, oxygen, sulfur, and carbon;
(b) wherein
if X is nitrogen, oxygen, or sulfur, then Y is carbon; and
if X′ is nitrogen, oxygen or sulfur, then Y′ is carbon; and
if Y is nitrogen, oxygen or sulfur, then X is carbon, and
if Y′ is nitrogen, oxygen or sulfur, then X′ is carbon; and
(c) wherein Z and Z′ taken together comprise a linking group, said linking group being selected from the following:
substituted aromatic, unsubstituted aromatic, substituted heteromatic, unsubstituted heteroaromatic, substituted polyaromatic or unsubstituted polyaromatic and
(d) wherein Q, W, D, and A independently may or may not be present, further wherein a maximum of two of Q, W, D, and A may be present in said compound, and
(e) wherein
if Q is present then D is not present; and
if W is present then A is not present; and
Q, W, D, and A always are attached to a carbon atom, and
Q, W, D, and A can be the same or different and are independently selected, and if present, may have the general structure 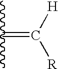
(f) wherein R may be selected from one or more of the following: C₁₋₂₀ alkyl, alkylester, halo, hydroxyl, hydrogen, cyano, sulfonyl, sulfato, nitro, carboxyl, C₁₋₂₀ carboxy, amino, C₁₋₂₀ alkylamino, acrylamino, C₁₋₂₀ alkylthio, C₁₋₂₀ alkylsulphonyl, C₁₋₂₀ alkylphenyl, phosphonyl, C₁₋₂₀ alkylphosphonyl, C₁₋₂₀ alkoxycarbonyl, arylamino, dialkylaminoaromatic, sulphonylamino, acyl, aryl, substituted aryl, heteroaryl, allyl, alkenyl, alkynyl, oxyalkylene, polyoxyalkylene, polyoxyalkylene substituted aromatic, azo, amide, ester, sulphonate, sulphonic acid, sulphonic acid salt, carboxylic acid salt, ether, benzyl, substituted amines, thio, phenylthio, thioethers, thioesters, silyl, siloxy, naphthyl, polyoxyalkyleneamino substituted aryl, polyoxyalkylene substituted aryl, substitiuted aromatics, aniline derivatives, 2,5-dimethoxyaniline derivatives, phenol derivatives, polyoxyalkylene substituted aniline derivatives, and polyoxyalkylene substituted phenol derivatives.

17. The compound of claim 16 where Z and Z′ taken together comprise a substituted or unsubstituted benzene ring

18. The compound of claim 16 where Z and Z′ taken together comprise a substituted or unsubstituted naphthalene ring.

19. The compound of claim 16 where Z and Z′ taken together comprise a substituted or unsubstituted anthraquinone ring.

20. The compound of claim 16 where Z and Z′ taken together comprise a substituted or unsubstituted anthracene ring.

21. The compound of claim 16 where Z and Z′ constitute a substituted or unsubstituted heteroaromatic ring system.

22. The compound of claim 16 where at least one of X, X′, Y, or Y′ comprises an oxygen atom.

23. The compound of claim 16 where at least one of X, X′, Y, or Y′ is an nitrogen atom.

24. The compound of claim 16 where at least one of X, X′, Y, or Y′ is an sulfur atom.

25. The compound of claim 16 wherein R comprises a substituted aromatic.

26. A plastic article comprising at least in part the composition of claim 16.

27. A method of synthesizing a bislactoarene (BLA) compound by employing in said synthesis at least one silyl enol ether intermediate.
